(19) United States
(12) Patent Application Publication
(10) Pub. No.: US 2009/0130103 A1
(43) Pub. Date: May 21, 2009

(54) PURIFICATION AND PROTECTIVE EFFICACY OF MONODISPERSE AND MODIFIED YERSINIA PESTIS CAPSULAR F1-V ANTIGEN FUSION PROTEINS FOR VACCINATION AGAINST PLAGUE

(75) Inventors: David F. NELLIS, Frederick, MD (US); Steven L. GIARDINA, Frederick, MD (US); Jeremy GOODIN, Aberdeen Proving Grounds, MD (US)

Correspondence Address: KLARQUIST SPARKMAN, LLP, 121 S.W. SALMON STREET, SUITE #1600, PORTLAND, OR, 97204-2988, US

(73) Assignee: The Government of the USA as represented by the Secretary of the Dept. of Health & Human Services

(21) Appl. No.: 11/944,230

(22) Filed: Nov. 21, 2007

(51) Int. Cl. A61K 39/00; C07K 16/00; C07H 21/04; C07K 1/14; C12N 15/00

(52) U.S. Cl. 424/134.1; 530/387.3; 536/23.4; 435/320.1; 530/416

(57) ABSTRACT

This disclosure concerns compositions and methods for the treatment and inhibition of infectious disease, particularly bubonic and pneumonic plague. In certain embodiments, the disclosure concerns immunogenic proteins, for instance substantially monodisperse F1-V fusion proteins, that are useful for inducing protective immunity against Y. pestis.

### FIELD OF THE DISCLOSURE

This disclosure concerns compositions and methods for the treatment and inhibition of infectious disease, particularly bubonic and pneumonic plague. In certain embodiments, the disclosure concerns immunogenic proteins, for instance monodisperse F1-V fusion proteins, that can be used to induce protective immunity against Y. pestis infection.

### BACKGROUND

Plague is an infectious disease caused by the bacteria Yersinia pestis, which is a non-motile, slow-growing facultative organism in the family Enterobacteriacea. Y. pestis is carried by rodents, particularly rats, and in the fleas that feed on them. Other animals and humans usually contract the bacteria directly from rodent or flea bites.

Yersinia pestis is found in animals throughout the world, most commonly in rats but occasionally in other wild animals, such as prairie dogs. Most cases of human plague are caused by bites of infected animals or the infected fleas that feed on them. Y. pestis can affect people in three different ways, and the resulting diseases are referred to as bubonic plague, septicemic plague, and pneumonic plague.

The World Health Organization statistics show that 2,118 cases of plague were diagnosed and reported in the year 2003 worldwide. Worldwide, there have been small plague outbreaks in Asia, Africa, and South America. Approximately 10 to 20 people in the United States develop plague each year from flea or rodent bites, primarily from infected prairie dogs-in rural areas of the southwestern United States. About one in seven of those infected die from the disease. There is also renewed concern about Yersinia pestis as an agent of bioterrorism, and that pneumonic plague could be used as a weapon via aerosol distribution. The Y. pestis bacterium is widely available in microbiology banks around the world, and thousands of scientists have worked with plague, making a biological attack a serious concern.

Killed whole vaccines against Y. pestis have been used since the 1890s (Williamson, (2001) J. Appl. Microbiol., 91:606-608). The whole-cell killed vaccine previously was available for people at possible high risk of exposure, such as military or laboratory personnel, but side effects were common, and multiple boosters were necessary. It also was unclear how well this vaccine protected against the pneumonic form of plague (Smego et al. (1999) Eur. J. Clin. Microbiol. Infect. Dis., 18:1-15). Therefore, production of the vaccine was discontinued by the manufacturer in 1999 (Inglesby et al. (2000) JAMA, 283:2281-2290). A live attenuated vaccine, EV76, also was in use in humans in some areas of the world, but it also is not commercially available (Williamson, (2001) J. Appl. Microbiol., 91:606-608). Previous experiments in mice revealed that purified Fl antigen was more effective in protecting against plague than the killed whole-cell vaccine (Friedlander et al. (1995) Clin. Infect. Dis., 21:5178-5181). However, attempts to develop a vaccine using only the Fl antigen were less than fully successful (Friedlander et al. (1995) Clin. Infect. Dis., 21:5178-5181).

A more efficacious vaccine was recently developed that includes a fusion protein of the Fraction 1 capsular antigen (F1, Caf1) with a second protective immunogen called the V-antigen (LcrV; Heath et al., (1998) Vaccine 16, 1131-1137). Although the F1-V vaccine provided better protection than the F1 vaccine, it tended to self-associate and form aggregates. Thus, the F1-V vaccine presented risks for large-scale manufacture including: 1) possible entrapment of contaminants within multimeric forms, which can lower process yields and increase process costs to achieve purity; and 2) uncontrolled or premature re-folding that can affect fusion-protein structure and thereby impact product consistency and long-term stability (Chi et al., (2003) Pharm. Res. 20, 1325-1336).

Given the foregoing, new and enhanced immunological compositions and methods for combating Yersinia pestis infection and disease are needed.

### SUMMARY OF THE DISCLOSURE

Disclosed herein is an improved F1-V vaccine that includes a substantially monodisperse immunogenic F1-V fusion protein. Unlike the previous F1-V fusion protein vaccine, the fusion protein described herein is substantially monomeric and does not tend to self-associate and form aggregates, yet it retains its immunogenicity.

Also disclosed are pharmaceutical compositions that include the substantially monodisperse immunogenic proteins as well as methods for eliciting an immune response in a subject, which methods include (a) selecting a subject in which an immune response to the substantially monodisperse immunogenic F1-V protein is desirable; and (b) administering to the subject a therapeutically effective amount of the substantially monodisperse immunogenic F1-V protein, thereby producing an immune response in the subject.

Further embodiments are methods of inhibiting Yersinia pestis infection in a subject. These methods include (a) selecting a subject at risk for exposure to Yersinia pestis; and (b) administering to the subject a therapeutically effective amount of a substantially monodisperse immunogenic F1-V protein, thereby inhibiting Yersinia pestis infection in the subject.

The foregoing and other features will become more apparent from the following detailed description of several embodiments, which proceeds with reference to the accompanying figures.

### BRIEF DESCRIPTION OF THE FIGURES

FIG. 1 includes several panels showing a diagram of an expression plasmid, primer sequences, and the amino acid sequence of the F1-V fusion protein. FIG. 1A is a diagram of the F1-V pET24a(+) Cys₄₂₅->Ser₄₂₅ expression plasmid. FIG. 1B shows the site-directed mutagenesis primers F1-V-CS-F and F1-V-CS-R that were used to produce the Cys->Ser mutation at amino acid position 424. FIG. 1C shows the complete F1-V fusion protein amino acid sequence, including the F1 Capsule Antigen (SEQ ID NO: 5), the Fusion Spacer (SEQ ID NO: 6), and the V-antigen (SEQ ID NO: 7). The sequences contain three marked points of interest: (1) the F1 capsule signaling/leader sequence, which is not included in the fusion; (2) an EcoRI restriction site that was used to link the F1 protein and V antigen, and that yields a two-amino acid (EF) linker between the F1 protein and the V antigen; and (3) the specific location of the Cys->Ser mutation within the fusion protein.

FIG. 2 is a digital image of a gel showing Simply Blue-stained (Invitrogen), reduced SDS-PAGE analysis of F1-V_{C424S} expression for whole broth soluble and insoluble fractions before (−) and after (+) IPTG induction. Lane M—protein molecular mass standard. The calculated molecular mass of F1-V_{C424S} monomer is 53 kDa. Cells and supernatants were mixed with 4×LDS sample buffer and heated at 70° C. for 10 minutes before electrophoresis through Invitrogen NuPAGE 4-12% Bis-Tris gels, using MOPS SDS running buffer.

FIG. 3 includes two panels showing the fermentation time course for cultivation of E. coli., BLR130 transformed with plasmid pW731 expressing F1-V (FIG. 3A; arrow, induction with 1 mM IPTG, 0.2% arabinose as described in Example 1), and Sypro Ruby-stained, reduced SDS-PAGE analysis of F1-V_{MN} recovery as described in Example 1 (FIG. 3B). Pellets were first prepared by ˜25-fold dilution into 2× reducing running buffer. Loadings were 20 μL/well or 40 μL/well (lanes 4, 5, and 6). Lanes: (1) washed initial pellet lysate; (2) Mark 12 molecular mass markers (Invitrogen); (3) lysate supernatant; (4) pH 4.8 supernatant; (5) 1^{st} pH 4.8 rinse; (6) 2^{nd} pH 4.8 rinse; (7) pH 4.8 pellet; (8) post resolubilization and pellet; (9) 2^{nd} pH 4.8 step supernatant; and (10) 2^{nd} pH 4.8 step pellet.

FIG. 4 is a series of graphs and digital images of gels showing F1-V_{MN} purification as described in Example 1. The grey boxes show the eluate pools. FIG. 4A shows the Q-Sepharose FF IEX profile. The dashed trace shows the eluate conductivity. A₂₅₄ (dotted trace) and A₂₈₀ (solid trace) were monitored across a 2-mm path length cell. The load and wash occurred before the elution started at 6 L. FIG. 4B shows the source 15Q IEX profile with 7 L elution start and 2-mm cell. FIG. 4C shows the CHT-T2 chromatography profile with 2 L elution start and 10-mm cell. FIG. 4D shows the preparative scale Superdex 200 PG SEC profile with 2% CV load and 10-mm cell. Three pools were made: a leading, highly-pure, dimer-enriched F1-V pool (open box); a central target F1-V_{MN} pool (light gray box); and a third trailing pool (dark box) containing monomeric F1-V_{MN} and a ˜49 kDa contaminant (asterisk). After re-concentration, the third pool was reprocessed through the Superdex 200 PG stage. The insets show Sypro-Ruby-stained, reduced 4-12% NuPAGE SDS-PAGE analysis from elution fractions.

FIG. 5 is a pair of digital images of gels and a graph showing SDS-PAGE analysis of final preparations. FIG. 5A (top) shows a comparison of F1-V_{MN} before and after conversion to F1-V_{AG}, loaded with two-fold dilution series starting at 9.2 μg/well; and FIG. 5A (bottom) shows F1-V_{C424S-MN} loaded starting at 9.6 μg/well. (FIG. 5A top, Lane 1, bottom Lane 5) Mark 12 MW marker. FIG. 5B shows an overlay of HPLC-SEC profiles of final F1-V_{MN} (pH 9.9), F1-V_{C424S-MN} (pH 9.9), and F1-V_{AG} (pH 5.0) preparations. F1-V monomer eluted at an anomalous apparent molecular size of 100 kDa relative to high MW size standards.

FIG. 6 is a pair of graphs showing an analysis of cysteine-stabilized F1-V_{MN} non-covalent self-association as described in Example 1. FIG. 6A shows stacked HPLC-SEC traces after incubation at 4° C. for 55-64 hours at pH 4.5-10.5. The ‘Adjustment Control’ sample was derived from the pH 4.5-sample that was immediately back-titrated to pH 10.0. Peak solution state assignments were based on SEC-MALLS MW determinations (FIG. 8). Peak A, F1-V monomer; Peak B, F1-V_{(S—S)} dimer (DTT-sensitive); Peak C, F1-V_{(NC)} dimer (DTT-insensitive); Peak D, F1-V trimer; Peak E, F1-V multimer less than 0.5 MDa; Peak F, multimer, 0.5 to 6 MDa. The L-cysteine-free control at pH 9.9 established the F1-V_{(S—S)} dimer position (Peak B). FIG. 6A, inset, shows a plot of the percentage of integrated peak area contained in dimer and multimer peaks as a function of incubation pH after 55-64 hours incubation. FIG. 6B shows related plots of the percent integrated peak area contained in dimer and multimer peaks as a function of time and pH between 0 and 64 hours incubation at 4° C.

FIG. 7 is a graph showing additive-induced F1-V non-covalent multimer-content modulation at pH 6.5 as described in Example 1.

FIG. 8 is a pair of graphs showing SEC-MALLS profiles for titrated F1-V_{MN} as described in Example 1. FIG. 8A shows the calculated molar masses as fitted squares (grey lines, peaks A′, B′, C′). Profiles were measured 0.5 hours after adjustment to pH 6.5 and, (black lines; peaks A, B, C, D) after 4 hours. Calculated peak molar masses with RI-based total protein determination; A′-55.2, A-52.0, B′-98.5, B-93.1, C′-101.8, C-102.2, D-167 kDa. Calculated peak molar masses with A280-based total protein determination; A-44.2, B-83.1, C-86.7, D 137.4 kDa. FIG. 8B shows profiles measured <4 hours after adjustment to pH 5.1 (grey, 20-μL and black 50-μL injections) of F1-V_{MN}. The light scattering maximum (peak E) preceded the protein content maximum (peak F) resulting in a range of calculated molar masses from 500 to >6,000 kDa.

FIG. 9 includes several panels showing the peptide mapping analysis of F1-V and F1-V_{C424S} preparations as described in Example 1. FIG. 9A shows a C₁₈ reverse phase-HPLC/MS base-peak profile overlay of tryptic digests for both preparations and a close-up showing elution positions for peptides corresponding to the native N-terminal fragment (residues 1-18, 25.7 minutes), modified N-terminus (+43 kDa, ˜28.4 minutes), two F1-V_{C424S}-derived fragments containing serine 424 (residues 398-427, 26.7 minutes and residues 406-438, 27.0 minutes), and a derived F1-V fragment containing cysteine 424 disulfide bonded to a single free L-cysteine (residues 398-427, predicted pre-adduct molecular mass 3,292.5 Da, plus 121.1 Da L-cysteine adduct, minus 2H from formation of disulfide bond, actual observed molecular mass=3,411.8 Da). The peak at 26.6 minutes was identified as an F1-V fragment (residues 306-340) unrelated to the F1-V_{C424S} derived peak at 26.7 minutes. FIG. 9B shows MS (Top) and MS/MS spectra for the F1-V derived native N-terminal fragment. FIG. 9C shows MS (Top) and MS/MS spectra for the F1-V derived modified N-terminal fragment. FIG. 9D shows MS (Top) and MS/MS spectra for a F1-V_{C424S}-derived fragment containing serine 424 (residues 398-427). FIG. 9E shows MS (Top) and MS/MS spectra for a F1-V_{C424S}-derived chymotryptic fragment containing serine 424 (residues 421-431, retention time=17.7 minutes, M+H=1162.5 Da). FIG. 9F shows MS (Top) and MS/MS spectra for the F1-V-derived fragment adducted to L-cysteine (residues 398-427+L-cysteine).

### SEQUENCE LISTING

The nucleic acid sequences listed in the accompanying sequence listing are shown using standard letter abbreviations for nucleotide bases, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the amino acid sequence of the immunogenic F1-V fusion protein F1-V_{C424X}.

SEQ ID NO: 2 is the amino acid sequence of the immunogenic F1-V fusion protein F1-V_{C424S}.

SEQ ID NO: 3 is a forward mutagenic primer

SEQ ID NO: 4 is a reverse mutagenic primer

SEQ ID NO: 5 is the amino acid sequence of an F1 capsule antigen.

SEQ ID NO: 6 is the amino acid sequence of a fusion spacer

SEQ ID NO: 7 is the amino acid sequence of a V-antigen.

### DETAILED DESCRIPTION

### I. Overview of Several Embodiments

Disclosed herein is an improved F1-V vaccine that includes a substantially monodisperse immunogenic F1-V fusion protein. Unlike the previous F1-V fusion protein vaccine, the fusion protein described herein is substantially monomeric and does not tend to self-associate and form aggregates, yet it retains its immunogenicity. Thus, one embodiment is an isolated immunogenic protein that includes a substantially monodisperse F1-V fusion protein. In some embodiments, the immunogenic protein includes about 50%, about 60%, about 70%, about 60%, about 80%, about 90%, or about 100% monodisperse F1-V fusion protein. In certain examples, the F1-V fusion protein includes either (A) the amino acid sequence set forth as SEQ ID NO: 1, wherein Xaa at position 424 is cysteine, methionine, serine, glycine, glutamic acid, aspartic acid, valine, threonine, tyrosine, or alanine; or (B) an amino acid sequence having at least 95% sequence identity with (a). In particular examples, the Xaa at position 424 is methionine, serine, glycine, glutamic acid, aspartic acid, valine, threonine, tyrosine, or alanine, and in other examples, the Xaa at position 424 is serine, and in yet other examples, the Xaa at position 150 is glutamic acid or asparagine. In even more particular examples, the Xaa at position 151 is phenylalanine, methionine, leucine, or tyrosine, while in other particular examples, the Xaa at position 150 is glutamic acid, and the Xaa at position 151 is phenylalanine.

In other embodiments, the immunogenic protein includes the amino acid sequence set forth as SEQ ID NO: 2, and in additional embodiments, the immunogenic protein is the amino acid sequence set forth as SEQ ID NO: 2. Other embodiments are isolated polynucleotides that include a nucleic acid sequence encoding the immunogenic protein, polynucleotides such as these operably linked to a promoter, vectors that include polynucleotides such as these, and the isolated immunogenic protein described above, wherein the protein provides protective immunity from Y. pestis when administered to a subject in a therapeutically effective amount.

Also disclosed are pharmaceutical compositions that include the immunogenic protein and a pharmaceutically acceptable carrier. In some embodiments, the composition is adsorbed to an aluminum hydroxide adjuvant, whereas in other embodiments, the composition includes from about 0.5 mM L-cysteine to about 5 mM L-cysteine. In still other embodiments, the composition includes from about 0.06 M L-arginine to about 6 M L-arginine, whereas in yet other embodiments, the composition also includes a therapeutically effective amount of IL-2, GM-CSF, TNF-α, IL-12, and IL-6.

Other embodiments are methods for eliciting an immune response in a subject. These methods include (a) selecting a subject in which an immune response to the immunogenic protein of claim 1 is desirable; and (b) administering to the subject a therapeutically effective amount of the immunogenic protein described above, thereby producing an immune response in the subject. In some examples of the method, administration includes oral, topical, mucosal, or parenteral administration, for instance intravenous administration, intramuscular administration, or subcutaneous administration. In other examples of the method, administration includes from about one to about six doses, for instance two doses. Still other examples of the method include administering an adjuvant to the subject, for instance a therapeutically effective amount of IL-2, RANTES, GM-CSF, TNF-α, IFN-γ, G-CSF or a combination thereof.

Still other embodiments include methods of inhibiting Yersinia pestis infection in a subject. These methods include (a) selecting a subject at risk for exposure to Yersinia pestis; and (b) administering to the subject a therapeutically effective amount of the immunogenic protein described above, thereby inhibiting Yersinia pestis infection in the subject.

Yet other methods are methods of making the isolated substantially monodisperse immunogenic protein described above. In some embodiments, these methods include ion exchange chromatography, wherein the ion exchange chromatography dilution buffer comprises guanidine HCl, for instance from about 3 M guanidine HCl to about 9 M guanidine HCl. In other embodiments of the method, the immunogenic protein is precipitated at a pH of about 4.7-5.2, and still other embodiments of the method further include raising the pH of the immunogenic protein to about 7.8-11.0. Particular examples of the method include hydroxyapatite chromatography, for instance ceramic hydroxyapatite or fluoroapatite chromatography.

### II. Abbreviations

˜ approximately
A_{X} absorbance at x nm.
ALH alhydrogel adjuvant
CHT ceramic hydroxyapatite chromatography
CL confidence limit
CV column volume
DTE dithioerythritol;
DTT dithiothreitol;
F1-V_{AG} multimer-enriched F1-V preparation derived from F1-V_{MN}
F1-V_{MN} monomer-enriched F1-V preparation (monodisperse)
F1-V_{C424S-MN} F1-V with cysteine 424 replaced with serine, monomer-enriched (monodisperse)
F1-V_{STD} previously reported preparation of F1-V
F1-V_{(S—S)} F1-V disulfide linked dimer
F1-V_{(NC)} F1-V non-covalently associated dimer
Gdn HCl guanidine hydrochloride
IAA iodoacetamide
IPTG isopropyl β-D-1-thiogalactopyranoside
HPLC-SEC high-performance liquid chromatography size-exclusion chromatography
IEX ion exchange chromatography
L-Cys L-cysteine
MOPS 3-(4-Morpholino)propane sulfonic acid
MW molecular weight
RANTES Regulated on Activation, Normal T Expressed and Secreted
RI refractive index
SDS sodium dodecyl sulfate
SEC-MALLS size exclusion chromatography-multi-angle laser light scattering

### III. Terms

In order to facilitate review of the various embodiments of the disclosure, the following explanations of specific terms are provided:

Adjuvant: An agent used to enhance antigenicity. Some adjuvants include a suspension of minerals (alum, aluminum hydroxide, or phosphate) on which antigen is adsorbed; or water-in-oil emulsion in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunostimulatory oligonucleotides (such as those including a CpG motif) can also be used as adjuvants (for example see U.S. Pat. No. 6,194,388; U.S. Pat. No. 6,207,646; U.S. Pat. No. 6,214,806; U.S. Pat. No. 6,218,371; U.S. Pat. No. 6,239,116; U.S. Pat. No. 6,339,068; U.S. Pat. No. 6,406,705; and U.S. Pat. No. 6,429,199). Adjuvants also can include biological molecules, such as costimulatory molecules. Exemplary adjuvants include IL-2, RANTES, GM-CSF, TNF-α, IFN-γ, G-CSF, LFA-3, CD72, B7-1, B7-2, OX-40L and 41 BBL. Adjuvants also can include dsRNA.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and 2-dimensional nuclear magnetic resonance.

Antibody: Immunoglobulin molecules and immunologically active portions of immunoglobulin molecules, for instance, molecules that contain an antigen binding site that specifically binds (immunoreacts with) an antigen.

A naturally occurring antibody (for example, IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the V_{L}, V_{H}, C_{L} and C_{H1} domains; (ii) an F_{d} fragment consisting of the V_{H} and C_{H1} domains; (iii) an Fv fragment consisting of the VL and VH domains of a single arm of an antibody, (iv) a dAb fragment (Ward et al., Nature 341:544-546, 1989) which consists of a V_{H} domain; (v) an isolated complimentarity determining region (CDR); and (vi) a F(ab′)₂ fragment, a bivalent fragment comprising two Fab fragments linked by a disulfide bridge at the hinge region.

Immunoglobulins and certain variants thereof are known and many have been prepared in recombinant cell culture (for instance, see U.S. Pat. No. 4,745,055; U.S. Pat. No. 4,444,487; WO 88/03565; EP 256,654; EP 120,694; EP 125,023; Faoulkner et al., (1982) Nature 298:286; Morrison, (1979) J. Immunol. 123:793; Morrison et al., (1984) Ann Rev. Immunol 2:239).

Animal: Living multi-cellular vertebrate organisms, a category that includes, for example, mammals and birds. The term mammal includes both human and non-human mammals. Similarly, the term “subject” includes both human and veterinary subjects.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Conservative variants: As used herein, the term “conservative variant,” in the context of an immunogenic F1-V fusion protein, refers to a peptide or amino acid sequence that deviates from another amino acid sequence only in the substitution of one or several amino acids for amino acids having similar biochemical properties (so-called conservative substitutions). Conservative amino acid substitutions are likely to have minimal impact on the activity of the resultant protein. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology. In some embodiments, conservative amino acid substitutions are those substitutions that do not substantially affect or decrease antigenicity of an immunogenic F1-V fusion protein. Specific, non-limiting examples of conservative substitutions include the following examples:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

In some embodiments, a conservative substitution or a cysteine residue can also include Met, Gly, Glu, Asp, Val, Thr, Tyr, or Ala. The term conservative variation also includes the use of a substituted amino acid in place of an unsubstituted parent amino acid, provided that antibodies raised to the substituted polypeptide also immunoreact with the unsubstituted polypeptide. Non-conservative substitutions are those that reduce antigenicity.

Epitope: An antigenic determinant. These are particular chemical groups or peptide sequences on a molecule that are antigenic (that elicit a specific immune response). An antibody specifically binds a particular antigenic epitope on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or 8 to 10 amino acids in a unique spatial conformation. Methods of determining spatial conformation of epitopes include, for example, x-ray crystallography and 2-dimensional nuclear magnetic resonance. See, for instance, “Epitope Mapping Protocols” in Methods in Molecular Biology, Vol. 66, Glenn E. Morris, Ed (1996).

Encode: As used herein, the term “encode” refers to any process whereby the information in a polymeric macromolecule or sequence is used to direct the production of a second molecule or sequence that is different from the first molecule or sequence. As used herein, the term is construed broadly, and can have a variety of applications. In some aspects, the term “encode” describes the process of semi-conservative DNA replication, where one strand of a double-stranded DNA molecule is used as a template to encode a newly synthesized complementary sister strand by a DNA-dependent DNA polymerase.

In another aspect, the term “encode” refers to any process whereby the information in one molecule is used to direct the production of a second molecule that has a different chemical nature from the first molecule. For example, a DNA molecule can encode an RNA molecule (for instance, by the process of transcription incorporating a DNA-dependent RNA polymerase enzyme). Also, an RNA molecule can encode a peptide, as in the process of translation. When used to describe the process of translation, the term “encode” also extends to the triplet codon that encodes an amino acid. In some examples, an RNA molecule can encode a DNA molecule, for instance, by the process of reverse transcription incorporating an RNA-dependent DNA polymerase. In another example, a DNA molecule can encode a peptide, where it is understood that “encode” as used in that case incorporates both the processes of transcription and translation.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (for instance, ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements that are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for instance, Bitter et al., (1987) Methods in Enzymology 153:516-544). For example, when cloning in bacterial systems, inducible promoters such as pL of bacteriophage lambda, plac, ptrp, ptac (ptrp-lac hybrid promoter) and the like can be used. In one embodiment, when cloning in mammalian cell systems, promoters derived from the genome of mammalian cells (such as the metallothionein promoter) or from mammalian viruses (such as the retrovirus long terminal repeat; the adenovirus late promoter; the vaccinia virus 7.5K promoter) can be used. Promoters produced by recombinant DNA or synthetic techniques can also be used to provide for transcription of the nucleic acid sequences.

Gene expression: The process by which the coded information of a nucleic acid transcriptional unit (including, for example, genomic DNA or cDNA) is converted into an operational, non-operational, or structural part of a cell, often including the synthesis of a protein. Gene expression can be influenced by external signals; for instance, exposure of a cell, tissue or subject to an agent that increases or decreases gene expression. Expression of a gene also can be regulated anywhere in the pathway from DNA to RNA to protein. Regulation of gene expression occurs, for instance, through controls acting on transcription, translation, RNA transport and processing, degradation of intermediary molecules such as mRNA, or through activation, inactivation, compartmentalization or degradation of specific protein molecules after they have been made, or by combinations thereof. Gene expression can be measured at the RNA level or the protein level and by any method known in the art, including, without limitation, Northern blot, RT-PCR, Western blot, or in vitro, in situ, or in vivo protein activity assay(s).

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. For example, an oligonucleotide can be complementary to an F1-V fusion protein-encoding RNA, or an F1-V fusion protein-encoding DNA.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

For purposes of the present disclosure, “stringent conditions” encompass conditions under which hybridization will only occur if there is less than 25% mismatch between the hybridization molecule and the target sequence. “Stringent conditions” can be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

In particular embodiments, stringent conditions are hybridization at 65° C. in 6×SSC, 5×Denhardt's solution, 0.5% SDS and 100 μg sheared salmon testes DNA, followed by 15-30 minute sequential washes at 65° C. in 2×SSC, 0.5% SDS, followed by 1×SSC, 0.5% SDS and finally 0.2×SSC, 0.5% SDS.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogenic protein: A protein that includes an allele-specific motif or other sequence such that the peptide will bind an MHC molecule and induce a cytotoxic T lymphocyte (“CTL”) response, or a B cell response (for instance, antibody production) against the antigen from which the immunogenic peptide is derived.

In one embodiment, immunogenic proteins are identified using sequence motifs or other methods, such as neural net or polynomial determinations, known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing protein. Within the context of an immunogenic protein, a “conserved residue” is one which appears in a significantly higher frequency than would be expected by random distribution at a particular position in a peptide. In one embodiment, a conserved residue is one where the MHC structure may provide a contact point with the immunogenic protein.

Immunogenic proteins also can be identified by measuring their binding to a specific MHC protein and by their ability to stimulate CD4 and/or CD8 when presented in the context of the MHC protein.

In one example, an immunogenic F1-V fusion protein is a series of contiguous amino acid residues from the F1 and V antigens that are connected by a short linker sequence. Generally, immunogenic immunogenic F1-V fusion proteins can be used to induce an immune response in a subject, such as a B cell response or a T cell response.

Immunogenic composition: A composition comprising an immunogenic F1-V fusion protein that induces a measurable CTL response against cells expressing PAGE4 polypeptide, or induces a measurable B cell response (such as production of antibodies that specifically bind the F1 and/or V antigens) against Y. pestis. For in vitro use, the immunogenic composition can consist of the immunogenic peptide alone. For in vivo use, the immunogenic composition will typically comprise the immunogenic polypeptide in a pharmaceutically acceptable carrier, and/or other agents. An immunogenic composition optionally can include an adjuvant, a costimulatory molecule, or a nucleic acid encoding a costimulatory molecule.

Isolated: An “isolated” biological component (such as a nucleic acid or protein or organelle) has been substantially separated or purified away from other biological components in the cell of the organism in which the component naturally occurs, for instance, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Linker sequence: A linker sequence is an amino acid sequence that covalently links two polypeptide domains. Linker sequences can be included in the between the F1 and V epitopes disclosed herein in order to provide rotational freedom to the linked polypeptide domains and thereby to promote proper domain folding and presentation to the MHC. By way of example, in a recombinant polypeptide comprising the F1 and V epitopes, a linker sequence can be provided between them. Linker sequences are generally between 1 and 12 amino acids in length.

Mammal: This term includes both human and non-human mammals. Similarly, the term “subject” includes both human and veterinary subjects.

Monodisperse: Refers to free-floating, unassociated, single protein molecules in a protein preparations, for instance unassociated F1-V single protein molecules, for instance at an intermediate frozen hold stage after SEC column purification. A monodisperse F1-V protein is an F1-V protein that, when analyzed by native HPLC-SEC/MALLS, has a major peak of the correct molecular weight (˜53 kDa). Refolded, purified, monodisperse F1-V protein can be stored as a substantially monodisperse preparation. A substantially monodisperse protein is, for instance, about 50% monodisperse, about 55% monodisperse, about 60% monodisperse, about 65% monodisperse, about 70% monodisperse, about 75% monodisperse, about 80% monodisperse, about 85% monodisperse, about 90% monodisperse, about 95% monodisperse, or about 100% monodisperse.

A “monodisperse” F1-V preparation is distinct from what is conventionally referred to as a “monomeric” F1-V preparation in that what is referred to in the scientific literature as a monomeric preparation generally is only transiently monomeric, whereas a monodisperse preparation remains substantially monomeric in storage and in use. In some instances, the term “monomeric” also is used in the literature to describe a lack of disulfide bridging, or a preparation that was initially an aggregate but that separated into a monomeric form on a gel, such as an SDS-PAGE gel.

Nucleic acid molecule: A polymeric form of nucleotides, which can include both sense and anti-sense strands of RNA, cDNA, genomic DNA, and synthetic forms and mixed polymers of the above. A nucleotide refers to a ribonucleotide, deoxynucleotide or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A nucleic acid molecule can include either or both naturally occurring and modified nucleotides linked together by naturally occurring and/or non-naturally occurring nucleotide linkages.

Nucleic acid molecules can be modified chemically or biochemically or can contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, peptides), intercalators (for example, acridine, psoralen, etc.), chelators, alkylators, and modified linkages (for example, alpha anomeric nucleic acids, etc.). The term “nucleic acid molecule” also includes any topological conformation, including single-stranded, double-stranded, partially duplexed, triplexed, hairpinned, circular and padlocked conformations.

Operably linked: A first nucleic acid sequence is operably linked with a second nucleic acid sequence when the first nucleic acid sequence is in a functional relationship with the second nucleic acid sequence. For instance, a promoter is operably linked to a coding sequence if the promoter affects the transcription or expression of the coding sequence. When recombinantly produced, operably linked nucleic acid sequences are generally contiguous and, where necessary to join two protein-coding regions, in the same reading frame. However, nucleic acids need not be contiguous to be operably linked.

Parenteral administration: administration by injection or infusion. Specific, non-limiting examples of parenteral routes of administration include: intravenous, intramuscular, intrathecal, intraventricular, intraarterial, intracardiac, subcutaneous, intradermal, intraperitoneal, epidural, intravitreal, and intraosseous infusion.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “therapeutically effective amount” is a quantity of a composition or a cell to achieve a desired effect in a subject being treated. For instance, this can be the amount necessary to induce an immune response in a subject. When administered to a subject, a dosage will generally be used that will achieve target tissue concentrations (for example, in 15 lymphocytes) that has been shown to achieve an in vitro effect.

Plague: An infectious disease caused by the bacteria Yersinia pestis, which is a non-motile, slow-growing facultative organism in the family Enterobacteriacea. Y. pestis is carried by rodents, particularly rats, and in the fleas that feed on them. Other animals and humans usually contract the bacteria directly from rodent or flea bites.

Yersinia pestis is found in animals throughout the world, most commonly in rats but occasionally in other wild animals, such as prairie dogs. Most cases of human plague are caused by bites of infected animals or the infected fleas that feed on them. Y. pestis can affect people in three different ways, and the resulting diseases are referred to as bubonic plague, septicemic plague, and pneumonic plague.

In bubonic plague, which is the most common form of Y. pestis-induced disease, bacteria infect the lymphatic system, which becomes inflamed. Bubonic plague is typically contracted by the bite of an infected flea or rodent. In rare cases, Y. pestis bacteria, from a piece of contaminated clothing or other material used by a person with plague, enter through an opening in the skin. Bubonic plague affects the lymph nodes, and within three to seven days of exposure to the bacteria, flu-like symptoms develop such as fever, headache, chills, weakness, and swollen, tender lymph glands (buboes). Bubonic plague is rarely spread from person to person.

Septicemic plague is contracted the same way as bubonic plague, usually through a flea or rodent bite, following which the bacteria multiply in the blood. However, septicemic plague is characterized by the occurrence of multiplying bacteria in the bloodstream, rather than just the lymph system. Septicemic plague usually occurs as a complication of untreated bubonic or pneumonic plague, and its symptoms include fever, chills, weakness, abdominal pain, shock, and bleeding underneath the skin or other organs. Buboes, however, do not develop in septicemic plague, and septicemic plague is rarely spread from person to person.

Pneumonic plague is the most serious form of plague and occurs when Y. pestis bacteria infect the lungs and cause pneumonia. Pneumonic plague can be contracted when Y. pestis bacteria are inhaled. Within one to three days of exposure to airborne droplets of pneumonic plague, fever, headache, weakness, rapid onset of pneumonia with shortness of breath, chest pain, cough, and sometimes bloody or watery sputum develop. This type of plague also can be spread from person to person when bubonic or septicemic plague goes untreated after the disease has spread to the lungs. At this point, the disease can be transmitted to someone else by Y. pestis-carrying respiratory droplets that are released into the air when the infected individual coughs.

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (for instance, a cDNA) independent of other sequences. The nucleotides can be ribonucleotides, deoxyribonucleotides, or modified forms of either nucleotide. The term includes single- and double-stranded forms of DNA.

Protein: Any chain of amino acids, regardless of length or post-translational modification (for instance, glycosylation or phosphorylation). In one embodiment, the protein is an F1-V fusion protein. With regard to proteins, “comprises” indicates that additional amino acid sequence or other molecules can be included in the molecule, “consists essentially of” indicates that additional amino acid sequences are not included in the molecule, but that other agents (such as labels or chemical compounds) can be included, and “consists of” indicates that additional amino acid sequences and additional agents are not included in the molecule.

Probes and primers: A probe comprises an isolated nucleic acid attached to a detectable label or reporter molecule. Primers are short nucleic acids, preferably DNA oligonucleotides, of about 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example by polymerase chain reaction (PCR) or other nucleic-acid amplification methods known in the art. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers can be selected that comprise about 20, 25, 30, 35, 40, 50 or more consecutive nucleotides.

Purified: The F1 and V epitopes and F1-V fusion proteins disclosed herein can be purified (and/or synthesized) by any of the means known in the art (see, for instance, Guide to Protein Purification, ed. Deutscher, Meth. Enzymol. 185, Academic Press, San Diego, 1990; and Scopes, Protein Purification: Principles and Practice, Springer Verlag, New York, 1982). Substantial purification denotes purification from other proteins or cellular components. A substantially purified protein is at least about 60%, 70%, 80%, 90%, 95%, 98% or 99% pure. Thus, in one specific, non-limiting example, a substantially purified protein is 90% free of other proteins or cellular components.

RANTES: A cytokine that is a member of the interleukin-8 superfamily of cytokines. RANTES is believed to be a selective attractant for memory T lymphocytes and monocytes. RANTES binds to CCR5 (a coreceptor of HIV).

Risk of exposure to Y. pestis: a subject is at “risk of exposure to Y. pestis” if there is an increased probability that the subject will be exposed to the bacterium relative to the general population. Accordingly, risk is a statistical concept based on empirical and/or actuarial data. Commonly, risk is correlated with one or more indicators, such as occupation, geographical location, living conditions, contact with rodents or fleas, or other occurrences, events or undertakings, of a subject. For example, with respect to risk of exposure to Y. pestis, indicators include but are not limited to military service and living conditions that expose the subject to rodents and fleas.

Sequence identity: The similarity between two nucleic acid sequences or between two amino acid sequences is expressed in terms of the level of sequence identity shared between the sequences. Sequence identity is typically expressed in terms of percentage identity; the higher the percentage, the more similar the two sequences. Methods for aligning sequences for comparison are described in detail below, in section IV B of the Detailed Description.

Subcutaneous administration: delivery, most often by injection, of an agent into the subcutis. The subcutis is the layer of tissue directly underlying the cutis, composed mainly of adipose tissue. Subcutaneous injections are given by injecting a fluid into the subcutis. Within the context of administering immunogenic F1-V proteins, subcutaneous administration most often will involve injection of an F1-V fusion protein with an acceptable carrier into the subcutis of a subject at risk of exposure to Y. pestis.

Therapeutically active polypeptide: An agent, such as an F1 or V epitope or an F1-V fusion protein that causes induction of an immune response, as measured by clinical response (for example increase in a population of immune cells, increased cytolytic activity against cells that express F1 or V, or protection from Y. pestis infection). In one embodiment, a therapeutically effective amount of an F1 or V epitope or an F1-V fusion protein is an amount used to generate an immune response against Y. pestis.

Vector: A nucleic acid molecule capable of transporting a non-vector nucleic acid sequence which has been introduced into the vector. One type of vector is a “plasmid,” which refers to a circular double-stranded DNA into which non-plasmid DNA segments can be ligated. Other vectors include cosmids, bacterial artificial chromosomes (BAC) and yeast artificial chromosomes (YAC). Another type of vector is a viral vector, wherein additional DNA segments can be ligated into all or part of the viral genome. Certain vectors are capable of autonomous replication in a host cell into which they are introduced (for example, vectors having a bacterial origin of replication replicate in bacteria hosts). Other vectors can be integrated into the genome of a host cell upon introduction into the host cell and are replicated along with the host genome. Some vectors contain expression control sequences (such as promoters) and are capable of directing the transcription of an expressible nucleic acid sequence that has been introduced into the vector. Such vectors are referred to as “expression vectors.” A vector can also include one or more selectable marker genes and/or genetic elements known in the art.

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

The singular terms “a,” “an,” and “the” include plural referents unless context clearly indicates otherwise. “Comprising” means “including.” “Comprising A or B” means “including A,” “including B” or “including A and B.” It is further to be understood that all base sizes or amino acid sizes, and all molecular weight or molecular mass values, given for nucleic acids or peptides are approximate, and are provided for description.

Suitable methods and materials for the practice or testing of the disclosure are described below. However, the provided materials, methods, and examples are illustrative only and are not intended to be limiting. Accordingly, except as otherwise noted, the methods and techniques of the present disclosure can be performed according to methods and materials similar or equivalent to those described and/or according to conventional methods well known in the art and as described in various general and more specific references that are cited and discussed throughout the present specification (see, for instance, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999).

IV. Purification and Protective Efficacy of Monodisperse and Modified Yersinia pestis Capsular F1-V Antigen Fusion Proteins for Vaccination Against Plague

A. Overview

Described herein is an improved vaccine that includes a fusion protein of the Fraction 1 capsular antigen (F1, Caf1) with a second protective immunogen called the V-antigen (an F1-V fusion protein), but that overcomes the tendency of the previous F1-V vaccine to self-associate and form covalently-linked aggregates, while also providing improved protection from Y. pestis.

The potential use of plague as a biological weapon has necessitated the continued development of effective prophylaxis. A previously licensed human plague vaccine (Plague Vaccine USP), consisting of killed whole-cell Yersinia pestis, protected against plague infection acquired subcutaneously (Titball & Williamson (2001) Vaccine 19, 4175-4184). However, this whole-cell vaccine was later shown to be ineffective against aerosol challenge and to be poorly protective against a virulent strain lacking capsule (Pitt et al., (1994) in Proceedings of the Abstracts of the 94^{th} General Meeting of the American Society for Microbiology, Washington, D.C.; Anderson et al., (1998) Am. J. Trop. Med. Hyg. 58, 793-799).

In an effort to produce a more efficacious vaccine, Heath et al. developed a recombinant vaccine composed of a fusion protein of the Fraction 1 capsular antigen (F1, Caf 1) with a second protective immunogen called the V-antigen (LcrV; Heath et al., (1998) Vaccine 16, 1131-1137). F1-V was originally purified using a polyhistidine tag, and the his(10)-F1-V vaccine protected experimental mice against pneumonic as well as bubonic plague produced by either F1⁺ or F1⁻ strains of Y. pestis (Heath et al., (1998) Vaccine 16, 1131-1137). As analyzed by a statistical comparison of potency (Powell et al., (2005) Biotechnol. Prog. 21, 1490-1510), the recombinant fusion-protein vaccine provided far better protection against the wild-type (F1⁺) strain than did the former Plague Vaccine USP, and it also showed a significant improvement in protection over a cocktail vaccine composed of the separate F1 and V antigens, as was first indicated after its creation (Anderson et al., (1998) Am. J. Trop. Med. Hyg. 58, 793-799; Powell et al., (2005) Biotechnol. Prog. 21, 1490-1510).

Based on the success of animal protection studies and with the intent to improve the fusion protein for product development, F1-V was subsequently re-engineered to remove the poly-histidine tag and placed under transcriptional control of the IPTG-inducible pET-24a expression system (plasmid pPW731) in Escherichia coli strain BL21 (DE3), and then purified from soft inclusion bodies using 6M urea and a two-column procedure including anion-exchange and hydrophobic interaction chromatography (Powell et al., (2005) Biotechnol. Prog. 21, 1490-1510). The untagged fusion protein showed equivalent immunogenicity and protective efficacy, and was less polydisperse in molecular structure than the individual F1 subcomponent, but still showed a tendency to aggregate under certain conditions. The tendency of F1-V to self-associate was revealed by analytical size exclusion chromatography (HPLC-SEC) coupled to multiple angle laser light scattering (called SEC-MALLS in combination), which clearly showed mixtures of monomer, dimer, and multimeric species of higher mass in all standard preparations of F1-V (Powell et al., (2005) Biotechnol. Prog. 21, 1490-1510).

During production, F1-V characteristically formed loose inclusion bodies—insoluble collections of protein—as expressed at high levels in E. coli (Powell et al., (2005) Biotechnol. Prog. 21, 1490-1510; Lee et al., (2006) Protein Sci. 15, 304-313; Panda (2003) Adv. Biochem. Eng. Biotechnol. 85, 43-93). Subsequently, inclusion body dispersal and re-association of F1-V by on-column refolding embodied a substantial effort for downstream processing, and solution-state heterogeneity (for instance, monomer, self-dimer, and self-multimer forms) persisted throughout chromatographic isolation of the target species. Thus, the prior technology presented risks for large-scale manufacture including: 1) possible entrapment of contaminants within multimeric forms, which can lower process yields and increase process costs to achieve purity; and 2) uncontrolled or premature re-folding that can affect fusion-protein structure and thereby impact product consistency and long-term stability (Chi et al., (2003) Pharm. Res. 20, 1325-1336).

With a view toward developing current good manufacturing practices-compliant F1-V manufacture, wherein final product purity and target protein structural definition are important for regulatory approval, described herein is a robust process for recovering monodisperse F1-V preparations that contain minimal self- and hetero-protein associated forms. This process addresses any uncertainty as to the comparative level of plague protection achievable using monomeric versus multimeric F1-V preparations. Concerns regarding ic F1-V plague vaccine efficacy are based upon prior haptan reports, where monodisperse antigens induced weaker immune responses than did protein assemblies (Miller et al., (1998) FEMS Immunol. Med. Microbiol. 21, 213-221), and the disease context in which F1 subunits are encountered as multimeric fiber structures (Zavialov et al., (2003) Cell 113, 587-596; Williams et al., (1972) J. Infect. Dis. 126, 235-241).

This specification discloses: 1) the development of a new purification scheme for isolation of true monomeric (monodisperse) F 1-V under reducing conditions (designated ‘F1-V_{MN}’); 2) the use of site-directed mutagenesis to substitute the sole cysteine in F1-V (C424) with serine (designated ‘F1-V_{C424S-MN}’), or with glycine, methionine, glutamic acid, aspartic acid, valine, threonine, tyrosine, or alanine to prevent disulfide dimer formation and to eliminate in-process reducing agents, oxygen exclusion, and reducing agent clearance; 3) recovery and purification of monomeric (monodisperse) F1-V_{C424S-MN} under atmospheric oxygen conditions; 4) characterization of the resulting F1-V_{MN} and F1-V_{C424S, MN} preparation solution states with respect to pH and stabilizing additives; 5) conversion of the F1-V_{MN} form to multimeric form (designated ‘F1-V_{AG}’ under controlled, low pH conditions; and 6) demonstration of vaccine protective efficacy against subcutaneous plague infection provided by an Alhydrogel-adsorbed, two-dose vaccination with F1-V_{C424S-MN}, F1-V_{MN}, and F1-V_{AG} forms compared to the previously reported standard F1-V preparation (designated F1-V_{STD}’; Powell et al., (2005) Biotechnol. Prog. 21 (2005), pp. 1490-1510). Vaccination with all F1-V forms tested resulted in significant, and essentially equivalent, protection against up to 10⁸ LD₅₀ of wild-type Y. pestis.

B. F1-V Fusion Proteins

Disclosed herein are improved F1-V vaccines that include an F1-V fusion protein designated “F1-V_{MN},” “F1-V_{C424X},” or in particular embodiments, “F1-V_{C424S}.” Unlike the previous F1-V fusion protein vaccine, the fusion protein described herein is substantially monomeric (monodisperse) and does not tend to self-associate and form aggregates, yet it retains its immunogenicity.

In addition to the specific improvements to the F1-V processing, purification, and vaccine formulations described below, in some embodiments, the improved vaccine was generated by replacing the cysteine at amino acid 424 of SEQ ID NO: 1 with another amino acid. Because this cysteine is located in a surface-accessible position when the crystal structure of the V antigen is examined, altering this amino acid potentially could have been detrimental to the antigenicity of the antigen. Substitution of this cysteine serves several functions. For instance, it eliminates the need for reducing agents during the processing of the protein and eliminates the covalent linkage problems associates with previous F1-V proteins.

The improved F1-V vaccines include the Fraction 1 capsular antigen (F1) with a modified version of a second protective immunogen called the V-antigen. The two antigens are separated by a short linker sequence. In one embodiment of the disclosure, the F1-V fusion protein is V_{C424X} (SEQ ID NO: 1), and the Xaa at position 424 is methionine, serine, glycine, glutamic acid, aspartic acid, valine, threonine, tyrosine, or alanine. In particular embodiments, the Xaa at position 424 is serine. This embodiment is referred to as F1-V_{C424S} (SEQ ID NO: 2). These substitutions result in a F1-V vaccine that provides excellent protective immunity against infection by Y. pestis. In addition, the F1-V fusion protein described herein is substantially monodisperse, which prevents the possible entrapment of contaminants within multimeric forms during manufacture of the vaccine, which can lower process yields and increase process costs to achieve purity. Furthermore, the monomeric (monodisperse) protein prevents uncontrolled or premature re-folding that can affect fusion-protein structure and thereby impact product consistency and long-term stability (Chi et al., (2003) Pharm. Res. 20, 1325-1336).

In addition to the substitutions described at amino acid 424, modifications can be made to the linker sequence at positions 150 and 151 in SEQ ID NOs: 1 and 2. For instance, the amino acid at position 150 is shown as aspartate in SEQ ID NO: 2, however in other embodiments, glutamic acid also can be substituted at this position. Likewise, although the amino acid in position 151 is shown as phenylalanine in SEQ ID NO: 2, in other embodiments, methionine, leucine, or tyrosine is substituted at this position. In addition to these amino acid variations, the length of the linker sequence also can be varied. For instance, it can include 1, 2, 3, or even more amino acids, so long as the fusion protein remains substantially monodisperse and provides therapeutically effective protective immunity from Y. pestis infection.

In addition to these changes, in some embodiments, F1-V_{C424X} variants include the substitution of one or several amino acids at positions other than those described above for amino acids having similar biochemical properties (so-called conservative substitutions). Conservative amino acid substitutions are likely to have minimal impact on the activity of the resultant protein. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology. In some examples, F1-V_{C424X} variants can have no more than 1, 2, 3, 5, or even 10 conservative amino acid changes. The following table shows exemplary conservative amino acid substitutions that can be made to an F1-V_{C424X} protein:

| Original Residue | Conservative Substitutions |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

C. Nucleic Acid Sequences and Variants

As any molecular biology textbook teaches, a peptide of interest is encoded by its corresponding nucleic acid sequence (for instance, an mRNA or genomic DNA). Accordingly, nucleic acid sequences encoding F1-V_{C424X} proteins are contemplated herein, at least, to make and use the F1-V_{C424X} proteins of the disclosed compositions and methods.

In one example, in vitro nucleic acid amplification (such as polymerase chain reaction (PCR)) can be utilized as a method for producing nucleic acid sequences encoding F1-V_{C424X} proteins. PCR is a standard technique, which is described, for instance, in PCR Protocols: A Guide to Methods and Applications (Innis et al., San Diego, Calif.: Academic Press, 1990), or PCR Protocols, Second Edition (Methods in Molecular Biology, Vol. 22, ed. by Bartlett and Stirling, Humana Press, 2003).

A representative technique for producing a nucleic acid sequence encoding an F1-V_{C424X} protein by PCR involves preparing a sample containing a target nucleic acid molecule that includes the F1-V_{C424X} nucleic acid sequence. For example, DNA or RNA (such as mRNA or total RNA) can serve as a suitable target nucleic acid molecule for PCR reactions. Optionally, the target nucleic acid molecule can be extracted from cells by any one of a variety of methods well known to those of ordinary skill in the art (for instance, Sambrook et al., Molecular Cloning: A Laboratory Manual, Cold Spring Harbor Laboratory Press, New York, 1989; Ausubel et al., Current Protocols in Molecular Biology, Greene Publ. Assoc. and Wiley-Intersciences, 1992). F1-V fusion proteins are expressed in a variety of cell types; for example, prokaryotic and eukaryotic cells. In examples where RNA is the initial target, the RNA is reverse transcribed (using one of a myriad of reverse transcriptases commonly known in the art) to produce a double-stranded template molecule for subsequent amplification. This particular method is known as reverse transcriptase (RT)-PCR. Representative methods and conditions for RT-PCR are described, for example, in Kawasaki et al. (In PCR Protocols, A Guide to Methods and Applications, Innis et al. (eds.), 21-27, Academic Press, Inc., San Diego, Calif., 1990).

The selection of amplification primers will be made according to the portion(s) of the target nucleic acid molecule that is to be amplified. In various embodiments, primers (typically, at least 10 consecutive nucleotides of an F1-V_{C424X} nucleic acid sequence) can be chosen to amplify all or part of an F1-V_{C424X}-encoding sequence. Variations in amplification conditions may be required to accommodate primers and amplicons of differing lengths and composition; such considerations are well known in the art and are discussed for instance in Innis et al. (PCR Protocols, A Guide to Methods and Applications, San Diego, Calif.: Academic Press, 1990). From a provided F1-V_{C424X} nucleic acid sequence, one skilled in the art can easily design many different primers that can successfully amplify all or part of a F1-V_{C424X}-encoding sequence.

As described herein, disclosed are nucleic acid sequences encoding F1-V_{C424X} proteins. Though particular nucleic acid sequences are disclosed herein, one of skill in the art will appreciate that also provided are many related sequences with the functions described herein, for instance, nucleic acid molecules encoding conservative variants of an F1-V_{C424X} disclosed herein. One indication that two nucleic acid molecules are closely related (for instance, are variants of one another) is sequence identity, a measure of similarity between two nucleic acid sequences or between two amino acid sequences expressed in terms of the level of sequence identity shared between the sequences. Sequence identity is typically expressed in terms of percentage identity; the higher the percentage, the more similar the two sequences.

Methods for aligning sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; Huang, et al., Computer Applications in the Biosciences 8:155-165, 1992; Pearson et al., Methods in Molecular Biology 24:307-331, 1994; Tatiana et al., (1999), FEMS Microbiol. Lett., 174:247-250, 1999. Altschul et al. present a detailed consideration of sequence-alignment methods and homology calculations (J. Mol. Biol. 215:403-410, 1990).

The National Center for Biotechnology Information (NCBI) Basic Local Alignment Search Tool (BLAST™, Altschul et al., J. Mol. Biol. 215:403-410, 1990) is available from several sources, including the National Center for Biotechnology Information (NCBI, Bethesda, Md.) and on the Internet, for use in connection with the sequence-analysis programs blastp, blastn, blastx, tblastn and tblastx. A description of how to determine sequence identity using this program is available on the internet under the help section for BLAST™.

For comparisons of amino acid sequences of greater than about 30 amino acids, the “Blast 2 sequences” function of the BLAST™ (Blastp) program is employed using the default BLOSUM62 matrix set to default parameters (cost to open a gap [default=5]; cost to extend a gap [default=2]; penalty for a mismatch [default=−3]; reward for a match [default=1]; expectation value (E) [default=10.0]; word size [default=3]; number of one-line descriptions (V) [default=100]; number of alignments to show (B) [default=100]). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 50%, at least 60%, at least 70%, at least 80%, at least 85%, at least 90%, at least 95%, at least 98%, or at least 99% sequence identity to the sequence of interest, for example the F1-V_{C424X} of interest.

For comparisons of nucleic acid sequences, the “Blast 2 sequences” function of the BLAST™ (Blastn) program is employed using the default BLOSUM62 matrix set to default parameters (cost to open a gap [default=11]; cost to extend a gap [default=1]; expectation value (E) [default=10.0]; word size [default=11]; number of one-line descriptions (V) [default=100]; number of alignments to show (B) [default=100]). Nucleic acid sequences with even greater similarity to the reference sequences will show increasing percentage identities when assessed by this method, such as at least 60%, at least 70%, at least 75%, at least 80%, at least 85%, at least 90%, at least 95%, r at least 98%, or at least 99% sequence identity to the F1-V_{C424X} of interest.

Another indication of sequence identity is hybridization. In certain embodiments, F1-V_{C424X} nucleic acid variants hybridize to a disclosed (or otherwise known) F1-V_{C424X}-encoding nucleic acid sequence, for example, under low stringency, high stringency, or very high stringency conditions. Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, although wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

The following are representative hybridization conditions and are not meant to be limiting.

Very High Stringency (detects sequences that
share at least 90% sequence identity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (detects sequences that share
at least 80% sequence identity)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (detects sequences that share
at least 50% sequence identity)

| | |
|---|---|
| Hybridization: | 6x SSC at RT to 55° C. for 16-20 hours |
| Wash at least | 2x-3x SSC at RT to 55° C. for 20-30 minutes each. |
| twice: | |

One of ordinary skill in the art will appreciate that F1-V_{C424X} nucleic acid sequences of various lengths are useful for a variety purposes, such as for use as F1-V_{C424X} probes and primers. In some embodiments, an oligonucleotide can include at least 15, at least 20, at least 23, at least 25, at least 30, at least 35, at least 40, at least 45, at least 50 or more consecutive nucleotides of an F1-V_{C424X} nucleic acid sequence. In other examples, F1-V_{C424X} oligonucleotides can be at least 50, at least 100, at least 150, at least 200, at least 250 or at least 300 consecutive nucleic acids of an F1-V_{C424X} nucleic acid sequence.

D. Therapeutic Methods and Pharmaceutical Compositions

A substantially monodisperse immunogenic F1-V fusion protein as disclosed herein can be administered to a subject in order to generate an immune response. In exemplary applications, the compositions are administered to a subject who is at risk for exposure to Yersinia pestis, who has been exposed to Y. pestis, or who has a Y. pestis infection, in an amount sufficient to raise an immune response to Y. pestis bacteria. Administration induces a sufficient immune response to inhibit infection with Y. pestis, slow the proliferation of the bacteria, inhibit their growth, or to reduce a sign or a symptom of a Y. pestis infection. Amounts effective for this use will depend upon the extent of exposure to Y. pestis bacteria, the route of entry of the bacteria into the body of the subject, the general state of the subject's health, and the robustness of the subject's immune system. A therapeutically effective amount of the compound is that which provides either an objectively identifiable improvement in resistance to infection with Y. pestis.

A substantially monodisperse immunogenic F1-V fusion protein can be administered by any means known to one of skill in the art (see Banga, “Parenteral Controlled Delivery of Therapeutic Peptides and Proteins,” in Therapeutic Peptides and Proteins, Technomic Publishing Co., Inc., Lancaster, Pa., 1995) either locally or systemically, such as by intramuscular, subcutaneous, or intravenous injection, but even oral, nasal, or anal administration is contemplated. In one embodiment, administration is by subcutaneous or intramuscular injection. To extend the time during which the protein is available to stimulate a response, the protein can be provided as an implant, an oily injection, or as a particulate system. The particulate system can be a microparticle, a microcapsule, a microsphere, a nanocapsule, or similar particle. (see, for instance, Banga, supra). A particulate carrier based on a synthetic polymer has been shown to act as an adjuvant to enhance the immune response, in addition to providing a controlled release. Aluminum salts can also be used as adjuvants to produce an immune response.

Optionally, one or more cytokines, such as interleukin (IL)-2, IL-6, IL-12, IL-15, RANTES, granulocyte macrophage colony stimulating factor (GM-CSF), tumor necrosis factor (TNF)-α, interferon (IFN)-α or IFN-γ, one or more growth factors, such as GM-CSF or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., (1998) J. Surg. Oncol. 68(2):122-38; Lotze et al., (2000), Cancer J Sci. Am. 6(Suppl 1):S61-6; Cao et al., (1998) Stem Cells 16(Suppl 1):251-60; Kuiper et al., (2000) Adv. Exp. Med. Biol. 465:381-90). These molecules can be administered systemically (or locally) to the host.

Some embodiments are pharmaceutical compositions including a substantially monodisperse immunogenic F1-V fusion protein is thus provided. In one specific embodiment, the pharmaceutical composition is adsorbed to aluminum hydroxide adjuvant (for instance, Alhydrogel, 1.3%; Superfos Biosector, Vedbaek, Denmark; 0.19 mg of aluminum per dose). In another embodiment, the pharmaceutical composition contains trace amounts of cysteine, for instance, from about 0.5 mM to about 5 mM L-cysteine. In yet another embodiment, the pharmaceutical composition includes, for instance, from about 0.6 M to about 6 M L-arginine.

In another embodiment, the immunogenic F1-V fusion protein is mixed with an adjuvant containing two or more of a stabilizing detergent, a micelle-forming agent, and an oil. Suitable stabilizing detergents, micelle-forming agents, and oils are detailed in U.S. Pat. No. 5,585,103; U.S. Pat. No. 5,709,860; U.S. Pat. No. 5,270,202; and U.S. Pat. No. 5,695,770. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, Del.), TWEEN 40™, TWEEN 20™, TWEEN 60™, ZWITTERGENT™ 3-12, TEEPOL HB7™, and SPAN 85™. These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, for instance, Schmolka, (1977) J. Am. Oil. Chem. Soc. 54:110, and Hunter et al., (1981) J. Immunol. 129:1244, PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, (1984) J. Immun. 133:3167. The agent can be provided in an effective amount, for example between 0.5 and 10%, or in an amount between 1.25 and 5%.

The oil included in the composition is chosen to promote the retention of the antigen in oil-in-water emulsion, for example, to provide a vehicle for the desired antigen, and preferably has a melting temperature of less than 65° C. such that emulsion is formed either at room temperature (about 20° C. to 25° C.), or once the temperature of the emulsion is brought down to room temperature. Examples of such oils include tetratetracontane and peanut oil or other vegetable oils. In one specific, non-limiting example, the oil is provided in an amount between 1 and 10%, or between 2.5 and 5%. The oil should be both biodegradable and biocompatible so that the body can break down the oil over time, and so that no adverse affects, such as granulomas, are evident upon use of the oil.

In one embodiment, the adjuvant is a mixture of stabilizing detergents, micelle-forming agent, and oil available under the name PROVAX® (IDEC Pharmaceuticals, San Diego, Calif.). An adjuvant can also be an immunostimulatory nucleic acid, such as a nucleic acid including a CpG motif, or a biological adjuvant (see above).

In one specific, non-limiting example, a pharmaceutical composition for intravenous administration would include about 0.1 mg to about 100 mg of substantially monodisperse, immunogenic F1-V protein per dose, for instance 10 mg, 15 mg, 20 mg, 25 mg, 30 mg, 40 mg, or 50 mg. Dosages from about 0.1 μg up to about 200 mg can be used, particularly if the agent is administered subcutaneously. Actual methods for preparing administrable compositions will be known or apparent to those skilled in the art and are described in more detail in such publications as Remingtons Pharmaceutical Sciences, 19^{th} Ed., Mack Publishing Company, Easton, Pa., 1995.

Single or multiple administrations of the compositions are administered depending on the dosage and frequency as required and tolerated by the subject. In one embodiment, the dosage is administered once as a bolus, but in another embodiment can be applied periodically until a therapeutic result is achieved. For instance, in one embodiment the vaccine is administered in at least two doses, for instance 3, 4, 5, or 6 or more, with the second and subsequent doses administered at least a week after the first dose, for instance, one month, two months, three months or six months or more after the first dose. Generally, the dose is sufficient to inhibit infection with Y. pestis without producing unacceptable toxicity to the subject.

E. Production of F1-V Fusion Proteins

The F1-V fusion proteins described herein are produced using specific modifications of conventional techniques. Generally, a nucleic acid encoding the F1-V fusion protein of interest is expressed in a host cell, such as a bacterial cell, the host cells are cultured, and the cells are harvested at the appropriate stage of growth. The F1-V fusion protein is then recovered from the cells using conventional techniques. In one specific, non-limiting example, the resulting cell paste is re-suspended in an appropriate buffer, for instance 50 mM Tris, 50 mM EDTA, pH 9.0, (without reducing agents), and is then homogenized, for instance at a backpressure of 10,000 to 15,000 psi. The homogenized paste is then clarified by centrifugation and the supernatant is collected.

F1-V is then precipitated, for instance by adjusting the pH to about 4.8, and the precipitate is collected by centrifugation. The pellet is then washed one or more times, for instance at about pH 4.8, and is stored below −70° C. The washed pellet, in some embodiments, is then re-suspended in solubilization buffer (for example, 10 mM Tris, 10 mM ethanolamine, 5 mM L-cysteine, 50 mM EDTA, pH 9.0) and mixed to disperse the pellet. The pH of the resulting solution is then adjusted, for instance, to about pH 11.0, and then to about pH 8.3. The F1-V-enriched supernatant is then separated from a lower density, colorless precipitate, and the supernatant is re-precipitated by slow adjustment to about pH 4.8 and stored below −70° C.

Following recovery of the F1-V fusion protein, in some embodiments, the protein is purified with Ion Exchange Chromatography (IEX) using conventional techniques with certain modifications. Briefly, in certain examples, the F1-V enriched pellet is re-suspended, for instance in 10 mM Tris, 10 mM ethanolamine, 10 mM Gdn HCL, pH 8.3, and then adjusted to pH 10.3, incubated, and re-adjusted to pH 8.3. High-purity solid urea is then added to obtain a concentration of 4.5 M urea and the solution is loaded onto Q-Sepharose FF resin equilibrated with, for instance, 10 mM Tris, 10 mM ethanolamine, 4.5 M urea, 10 mM Gdn HCl, pH 8.3. This is followed by washing and linear gradient elution to 3.5 M urea/500 mM Gdn HCl at 120 cm/hour. The leading shoulder of a complex multi-peak structure is excluded, and F1-V monomer-enriched fractions are collected and pooled from the first major peak eluting between 40 and 80 mM chloride, and stored below −70° C. This s then diluted to 3.4 mS/cm (˜2.5-fold), loaded onto Source 15Q resin equilibrated with IEX-A buffer, and eluted with a linear gradient to 40% B over 16 CV at 120 cm/h (4 ml/min). The leading half of the main peak is then pooled and stored below −70° C.

In some embodiments, after IEX, the resulting fraction is further purified using ceramic hydroxyapatite chromatography (CHT affinity chromatography) using conventional techniques. Briefly, in some embodiments, CHT-T1 resin is equilibrated and developed by charging with high phosphate buffer and equilibrated CHT-A buffer (10 mM Tris, 150 mM NaCl, 1 mM NaH₂PO₄, 0.1 mM CaCl₂, pH 7.8; argon sparged; 1 mM DTE added). The sample is then thawed and processed through, for instance, two CHT-T1 column cycles. Generally, the resulting fractions are stored chilled.

Following CHT affinity chromatography, in some embodiments the fractions are then subjected to size exclusion chromatography (SEC). In general, the fractions were pooled and adjusted to 500 mM L-arginine. After 10 minutes at pH 11.0, the pool is adjusted to pH 10.1 and held overnight at 4° C. The adjusted pool is then fractionated by size-exclusion chromatography through Superdex 200 PG resin, and equilibrated and developed with 20 mM L-arginine, 10 mM NaCl, pH 10.0 (with no L-cysteine). Fractions in the first half of the monomer peak are generally pooled and stored below −70° C., thawed, concentrated at 4° C., filtered, distributed into sterile cryo-vials, and stored below −70° C.

Optionally, after purification, the total protein content of the F1-V fractions can be determined using conventional techniques, and/or the protein can be lyophilized.

The following examples are provided to illustrate certain particular features and/or embodiments. These examples should not be construed to limit the disclosure to the particular features or embodiments described.

### EXAMPLES

### Example 1

### Materials and Methods

This Example describes materials and methods that were used in performing Examples 2-16, below. Although particular methods are described, one of skill in the art will understand that other, similar methods also can be used.

### Bacterial Strain, Plasmid Construction, Cultivation, and Induction

For F1-V_{MN} the E. coli strain BLR130 and the F1-V expression vector, pPW731 (USAMRIID), controlled under a T7 promoter, were used for F1-V expression (Powell et al., (2005) Biotechnol. Prog. 21 (2005), pp. 1490-1510). A growth medium of soytone, yeast extract, and glucose (J. T. Baker, Phillipsburg, N.J.) and the antibiotics kanamycin (30 mg/L) and tetracycline (15 mg/L; Sigma, St. Louis, Mo.) were used in phosphate buffer, pH ˜7.3. Sterile medium in shaker flasks (300 ml) was inoculated with 1 ml of the strain from a previously made glycerol stock and incubated for ˜13 hours at 37° C. with shaking at 220 rpm. Batch cultivations were carried out in a Bioflo 4500 (New England Biolabs, Ipswich, Mass.) equipped with a 15-L vessel and 10-L working volume. Growth medium (9.7 L) was inoculated with 300 ml of seed culture. The dissolved oxygen concentration was maintained above 15% air saturation at 37° C. by controlling the aeration and agitation rates through BIOCOMMAND software (New England Biolabs). Solution pH was kept between 7.2 and 7.4 by adding 0.1 N HCl or 30% NH₄OH. After 3.5 hours, the culture was induced with IPTG (1 mM) and harvested 2 hours later by centrifugation. Cell paste aliquots were stored below −70° C.

For F1-V_{C425S}, TOP10, BL21 (DE3), and BL21 Star (DE3) E. coli strains were from Invitrogen (Carlsbad, Calif.). BL21 Star cells carried a mutated rne gene that encoded a truncated RNase E protein lacking the capacity to degrade mRNA and leading to increased mRNA stability and enhanced protein expression. The F1-V pET24a(+) Cys₄₂₅→Ser₄₂₅ expression plasmid (F1-V_{C424S}) was prepared by site-directed mutagenesis of the original cysteine-containing caf1-lcrV gene fusion (expressing F1-V_{STD}) on source plasmid F1-V_{STD} pET-24a (pPW731) (Powell et al., (2005) Biotechnol. Prog. 21 (2005), pp. 1490-1510). Site-directed mutagenesis was performed with the Quick-change site-directed mutagenesis kit (Stratagene, La Jolla, Calif.). Complementary mutagenic primers F1-V-CS-F (5′-CT CAC TTT GCC ACC ACC TCC TCG GAT AAG TCC AGG CCG C-3′; SEQ ID NO: 1) and F1-V-CS-R (5′-GCG GCC TGG ACT TAT CCG AGG AGG TGG TGG CAA AGT GAG-3′; SEQ ID NO: 2) were constructed with consideration of primer length (39 bp), % GC (59%), and melting temperature (85.8° C.). Each primer (125 ng) was combined with 50 ng of F1-V pET-24a (pPW731), along with the additional reaction chemistry as recommended by the manufacturer. Cycling parameters for the mutagenesis reaction included one cycle of 95° C. for 30 seconds, followed by 12 cycles of 95° C. melting for 30 seconds, 53° C. annealing for 1 minute, and 68° C. extension for 7 minutes, concluding with a 4° C. hold. The mutagenesis reaction was then digested with 1 μl of DpnI at 37° C. for 1 hour. The DpnI-digested mutagenesis reaction (1 μL) was used to transform chemically competent TOP10 E. coli, and the transformed cells were grown on LB plates containing 50 μg/ml kanamycin. Positive clones were verified by bidirectional DNA sequence analysis on an ABI 3100 genetic analyzer (Applied Biosystems, Foster City, Calif.). The F1-V_{C424S} vector was transformed into BL21 Star cells for protein expression under control of the isopropyl-β-D-thiogalacto-pyranoside (IPTG)-inducible T7 promoter. F1-V_{C424S}-BL21 Star E. coli starter cultures were grown overnight in four 4-L shaker-flasks filled with a total of 10-L LB medium at 37° C., and 250-rpm shaking in the presence of 50 mg/L of kanamycin. Starter cultures were then diluted 1:10 in fresh kanamycin-supplemented LB medium and grown at 37° C., 250 rpm to an OD₆₀₀ of 0.5-0.8. Protein expression was induced by adding IPTG (0.5 mM). After 3 hours at 37° C. with 250-rpm shaking, cell pellets were collected by centrifugation at 10,000×g for 20 minutes and stored at −70° C.

### Recovery of F1-V_{MN}

For F1-V_{MN}, combined wet cell paste from two fermentations was re-suspended to 40% w/v with 1.2 L of lysis buffer (50 mM Tris, 50 mM EDTA, 20 mM DTE, pH 9.0), sheared for 10 minutes with a HAAKE A82 (Thermo-Electron, Waltham, Mass.), and homogenized by three passages at 12,000 psi through a NS1001-L2K mechanical homogenizer (Niro-Soavi, S.p.A., Parma, Italy). The homogenizer was fitted with a chilled reservoir and cooling coil that was kept below 11° C. The homogenized paste was adjusted to pH 8.3+/−0.2, clarified by centrifugation for 1 hour at 10,000 RPM in a JA-10 rotor at 4° C. (Beckman Coulter, Fullerton, Calif.), and the supernatant was collected. F1-V was precipitated by a slow, well-mixed adjustment of the supernatant to pH 4.8 with 1 M acetic acid (pH 2.25). An off-white, granular pellet, enriched in F1-V, was collected by centrifugation for 1 hour. The pellet was washed in an equal volume of 5 mM citric acid, pH 4.8 and then centrifuged, washed again, and the resulting pellet stored below −70° C. The washed pellet was re-suspended in 2.5 volumes (˜1 L) of solubilization buffer (10 mM Tris, 10 mM ethanolamine, 5 mM L-cysteine, 50 mM EDTA, pH 9.0) and mixed to disperse the pellet at 20° C. for 20 minutes. The solution was adjusted to pH 11.0 by a slow, drop-wise addition of 10 N NaOH and held for 5 minutes at 20° C., then adjusted to pH 8.3 with vigorous mixing and slow addition of 1 M acetic acid. The F1-V-enriched supernatant was separated from a lower density, colorless precipitate, enriched in contaminants (notably 40 kDa E. coli membrane protein I, identified by N-terminal sequencing) by centrifugation. The supernatant was re-precipitated by slow adjustment to pH 4.8 and the F1-V enriched pellet was stored below −70° C.

For F1-V_{C424S-MN}, cell paste was re-suspended to 20% w/v with 50 mM Tris, 50 mM EDTA, pH 9.0, (without reducing agents) and homogenized by three passages through an EmulsiFlex-C5 MicroFluidizer (Avestin, Canada) at a backpressure of 10,000 to 15,000 psi. The homogenized paste was clarified by centrifugation for 35 minutes at 15,000 rpm in an SS-34 rotor at 4° C. (Beckman Coulter, Fullerton, Calif.). Using methods similar to those used for F1-V, except without the addition of reducing agents, F1-V_{C424S} was recovered from the supernatant. The recovered F1-V_{C424S}-enriched pellet was stored below −70° C.

### Initial IEX

Columns and chromatography systems were cleaned and depyrogenated by exposure to 0.05 N NaOH for greater than 12 hours or 0.5 N NaOH for 1 hour followed by rinsing to neutral pH. For F1-V_{MN}, the F1-V-enriched pellet (400-g) was thawed at 20° C. and re-suspended 1:10 into 4 L of IEX-A buffer (10 mM Tris, 10 mM ethanolamine, 4.5 M urea, pH 8.3; then nitrogen sparged; and 5 mM fresh L-cysteine was added). The load (˜2.9 mS/cm) was held at 20° C. for ˜3 hours for F1-V dispersal and applied onto Q-Sepharose FF resin (BPG100/500, 10 cm D×20 cm H bed, 90-μm bead size; GE Healthcare, Piscataway, N.J.) and developed with one CV rinse and six CV linear gradient elutions at 60 cm/hour to 3.5 M urea, 500 mM Gdn HCl in similar buffer (IEX-B). Monomer-enriched fractions, identified by HPLC-SEC analysis, were examined by SDS-PAGE to facilitate selection of the target monomeric (monodisperse) F1-V species. The first major F1-V elution peak was collected between the 80- to 130-mM chloride ion (6.0 to 9.7 mS/cm) range. The Q-Sepharose FF elution pool was stored below −70° C.

For F1-V_{C424S-MN}, the F1-V_{C424S} enriched pellet was re-suspended to 20-ml final volume with 10 mM Tris, 10 mM ethanolamine, 10 mM Gdn HCL, pH 8.3, and then adjusted to pH 10.3, held for 30 minutes, and re-adjusted to pH 8.3 with 1 M acetic acid. High-purity solid urea was added to obtain a concentration of 4.5 M urea and the solution was held at 20° C. for 1 to 2 hours before being loaded onto Q-Sepharose FF resin (1.6×10 cm, 90-μm bead size; GE Healthcare) equilibrated with 10 mM Tris, 10 mM ethanolamine, 4.5 M urea, 10 mM Gdn HCl, pH 8.3, followed by washing and linear gradient elution to 3.5 M urea/500 mM Gdn HCl at 120 cm/hour. The leading shoulder of a complex multi-peak structure was excluded from pooling to eliminate contaminants, identified by SDS-PAGE fraction analysis. F1-V_{C424S} monomer-enriched fractions were collected and pooled from the first major peak eluting between 40 and 80 mM chloride, and stored below −70° C. The second half (85 mg), was pooled separately and not processed further. The Q-Sepharose FF pool was diluted with high-quality water to 3.4 mS/cm (˜2.5-fold), loaded onto Source 15Q resin (1.6×10 cm, 15-μm bead size, GE Healthcare) equilibrated with IEX-A buffer, and eluted with a linear gradient to 40% B over 16 CV at 120 cm/h (4 ml/min). The leading half of the main peak was pooled and stored below −70° C.

For F1-V_{MN}, buffers IEX-A and IEX-B were made as above except for replacement of L-cysteine with 1 mM DTT. To ensure complete protein reduction, DTT (5 mM) was added to the monomer-enriched pool. After 2.3-fold dilution (from ˜9.5 mS/cm to 4.2 mS/cm, 4.75 L final volume) with IEX-A buffer, the pool was loaded onto Source 15Q resin (BPG100/500, 10 cm D×20 cm H, 15-μm bead size; GE Healthcare), and eluted with a linear gradient to 40% IEX-B over eight CV at 60 cm/hour. The F1-V monomer, eluting below 100 mM chloride ion, was pooled based on HPLC-SEC and SDS-PAGE fraction analysis. Contaminants present in a leading shoulder of a complex multi-peak structure were excluded from pooling. Two trailing shoulders, while also containing F1-V, were pooled separately and not processed further. The Source 15Q Elution Pool was separated into 5×200 mL aliquots and stored below −70° C.

### CHT Affinity Chromatography

For F1-V_{MN}, CHT Type 2 resin (BPG100/500, 10 cm×12 cm, 20-μm beads, BioRad, Hercules, Calif.), was charged with high phosphate buffer and equilibrated just before use with CHT-A buffer (10 mM Tris, 150 mM NaCl, 1 mM NaH₂PO₄, 0.1 mM CaCl₂, pH 7.8; argon sparged; 1 mM DTE added, used immediately). For each of five CHT-T2 cycles, a 200 mL Source 15Q Elution Pool aliquot was thawed at ˜20° C., adjusted to 1 mM NaH₂PO₄, 0.1 mM CaCl₂, from 100 mM stocks, diluted fivefold into CHT-A buffer, applied to the column at 50 cm/hour and eluted with a linear gradient to 50% Buffer CHT-B (CHT-A+200 mM NaH₂PO₄) over 16 CV. CHT T2 elution fractions were collected into containers pre-loaded with L-arginine stock (1.3 M L-arginine, pH 10.0) to obtain a final concentration of 200 mM L-arginine in each collected fraction. An early-eluting, sharp, F1-V-containing peak was excluded from pooling. Center fractions within a broader major peak were pooled and concentrated to 8 to 9 mg/ml of total protein by A₂₈₀ over a 1-ft² PrepScale-TFF 10-kDa MW cut off spiral tangential flow filtration membrane (regenerated cellulose, Cat# CDUF001LG; Millipore, Billerica, Mass.). The concentrated (7.4 mg/ml) CHT-T2 pool was divided into 3×95 mL aliquots and stored below −70° C.

For F1-V_{C424S (MN)}, CHT-T1 resin (1.6×10 cm, 20-μm bead size; BioRad, Hercules, Calif.) was equilibrated and developed similarly to CHT Type 2 resin above. The Source 15Q pool was thawed and processed through two CHT-T1 column cycles. During the first cycle, performed without trace phosphate added to the load, a portion of F1-V_{C424S} did not bind. For the second cycle, 1 mM phosphate was added to the load, leading to complete F1-V_{C424S} binding. For both cycles a single, notably sharp, concentrated elution peak, was pooled with a minor, extended tail excluded. The fractions were stored chilled.

### Size Exclusion Chromatography Formulation of F1-V_{MN}

Each CHT-T2 aliquot (1.2% CV) was adjusted to pH 10.0, held overnight at 4° C., loaded onto Superdex 200 PG resin (10 cm×90 cm in a BPG 100/950 column, 34-μm bead size) and eluted with formulation buffer (20 mM L-arginine, 10 mM NaCl, argon, 1 ml of L-cysteine, pH 10.0) at a flow rate of 22 cm/hour. The early eluting dimer-enriched fractions were pooled separately (252 mg) and stored below −70° C. Fractions in the first half of the monomer peak, essentially free of contaminants, were 0.2-μm filtered, aliquoted, and stored below −70° C. Trailing monomer-peak fractions, enriched in contaminants, were concentrated as above, re-fractionated, and combined with initial monomer-enriched fractions. This final pool was 0.2-μm filtered distributed into sterile cryo-vials; and stored below −70° C.

For F1-V_{C424S-MN}, main peak fractions were pooled (40 ml) and adjusted to 500 mM L-arginine by adding 3.5 g of solid L-arginine pre-dissolved in 7 ml water. After 10 minutes at pH 11.0, the pool was adjusted to pH 10.1 by the slow addition of HCl and held overnight at 4° C. This yielded ˜47 ml at 5.0 mg/mL or 235 mg of total protein. The adjusted CHT-T1 pool was fractionated by size-exclusion chromatography through Superdex 200 PG resin (two tandem columns, 10 cm×90 cm in BPG 100/950 columns, 34-μm bead size), equilibrated and developed with 20 mM L-arginine, 10 mM NaCl, pH 10.0 (with no L-cysteine) at a flow rate of 22 cm/hour. A 43-ml sample (0.3% of CV) was applied. Fractions in the first half of the monomer peak were pooled and stored below −70° C.; thawed; concentrated using YM-10 Centripreps (Millipore, Billerica, Mass.) at 4° C.; 0.2-μm filtered; distributed into sterile cryo-vials; and stored below −70° C.

### Conversion of Monomeric F1-V_{MN} to Multimeric F1-V_{AG}

An aliquot of formulated F1-V_{MN}, at pH 10.0, was converted to F1-V_{AG} by slow titration with acetic acid to pH 5.1, incubated overnight at 4° C., and then stored below −70° C.

### Optional Freeze-Drying of F1-V_{MN}

F1-V in formulation buffer was adjusted to 2% w/v low endotoxin D-mannitol (Ferro Phanstiehl Laboratories, Inc., Waukegan, Ill.) added from a 20% D-mannitol stock dissolved in formulation buffer. The product was distributed into 3-ml glass vials, frozen at a plate temperature of −48° C., and lyophilized in an AdVantange-ES Benchtop freeze-dryer (VirTis, Gardiner, N.Y.) for 30 hours at −45° C., 8 hours at −37° C., followed by 15 hours at +37° C. Condenser coils were maintained at −80° C. Vial stoppers were mechanically seated within the chamber while under vacuum and crimped externally. The vials were stored below −70° C.

### Total Protein, Endotoxin and SDS-PAGE

Protein concentrations were measured by A₂₈₀ divided by an absorption co-efficient of E=0.468 A_{280, 1cm} per (mg of F1-V/ml), calculated using methods (Pace et al., (1995) Protein Sci. 11, pp. 2411-2423) automated on the ExPASy Proteomic Server, ProtParm (2005 version). For solubilized pellets, total protein was estimated with E=1.0. For endotoxin measurement, the commercially available Charles River (Charleston, S.C.) kinetic chromogenic limulus amoebocyte lysate reactivity endotoxin kit was used, which had a lower detection limit of 0.005 EU/ml, established versus the provided endotoxin standard. For SDS-PAGE, 4-12% Bis-Tris NuPAGE gels and reagents, Mark 12 size standards, and Sypro Ruby fluorescent stain were obtained from Invitrogen. Samples were reduced with 5% v/v 2-mercaptoethanol. Destained gels were scanned with a Molecular Dynamics model 595 scanning laser fluorimeter (GE Healthcare) and integrated with ImageMaster ID Elite software (Version 4.1, GE Healthcare).

### SEC-MALLS

Size-exclusion chromatography coupled to multiangle laser light scattering (SEC-MALLS) was applied as previously reported for F1-V (Powell et al., (2005) Biotechnol. Prog. 21 (2005), pp. 1490-1510), but with modifications as published (Gidh et al, (2006) J. Chromatogr. A. 1114, pp. 102-110; Casini et al., (2004) Virology 325, pp. 320-327). The HPLC pumps were Rainin HPXL 10 ml/minute pumps (Varian, Walnut, Calif.) run at 0.4 ml/minute. Fractionation was performed through two tandem G3000SWxl analytical size-exclusion chromatography columns (7.8×250 mm, 5-μM bead size, 250-Å pore size; Tosho Biosciences, Montgomeryville, Pa.), equilibrated with 0.2-μm filtered, helium-sparged mobile phase (0.1 M KH₂PO₄, 0.1 M Na₂SO₄, 0.3 M NaCl, pH 7.0). The light-scattering detector series consisted of a Rainin Dynamax UV-1 A₂₈₀ detector (Varian); a Dawn EOS multi-angle, static, light-scattering detector (Wyatt Technology Corporation, Santa Barbara, Calif.); and an Optilab DSP interferometric refractometer (Wyatt). Average molar mass measurements were determined from aligned elution profiles within ASTRA for Windows software (Revision 4.90.07/QELS version 1.00, Wyatt). Bovine serum albumin (2 mg/ml, 10 μL injections) containing a mixture of solution forms (66.3 kDa monomer, 132.6 dimer kDa, and 198.9-kDa trimer; Pierce-Endogen, Rockford, Ill.) was used to normalize detectors, establish detector train delay times, set software parameters, and confirm system suitability before test sample analysis. According to previously reported methods (Casini et al., (2004) Virology 325, pp. 320-327), the standard optical constant was calculated as K*=1.85×10⁻⁷ mol cm² g⁻²; as derived from (dn/dc)=0.185 ml g⁻¹, n₀=1.33; and λ₀=681 nm; with the form factor set to unity.

### Peptide Mapping

For each sample, 100-μg aliquots were dried, re-solubilized to 1 M Gdn HCl in 0.1 M Tris, pH 8.0, divided in half and digested (1:30 enzyme-to-substrate ratio) with modified trypsin or chymotrypsin overnight at 37° C. with mixing by vortex at 1,200 rpm. The digest was quenched by acidification and the samples stored at 4° C. until analysis. The samples were injected onto a reverse-phase column (Grace Vydac LC/MS C18, 2.1×250 mm, C/N 218MS52, 35° C.; Hesperia, Calif.) fitted to an HPLC (Thermo Electron, Surveyor LC System, Waltham, Mass.) followed by a hold at 5% for 5 minutes and elution over 55 minutes at 0.2 ml/minute using a 1% per minute linear gradient of acetonitrile containing 0.08% trifluoroacetic acid and 0.02% formic acid with elution monitored at 214 nm. The effluent was directed into an ion trap mass spectrometer (Thermo Electron, LCQ-Deca MS) for detection by electrospray mass spectrometry (ESI-MS) in positive mode ionization with 250° C. capillary temperature, ˜95 psi sheath gas pressure, ˜5 psi auxiliary gas pressure, source at 5.5 kV with capillary at 44 V, lens offset by 50 V, multipole offset by −5.5 and −10.5V, inter multipole lens at −28V, entrance lens at −88V and a trap DC offset of −10V. MS/MS was performed using 35% collision energy. Sequential scanning, consisting of full-scan ESI-MS from m/z 500 to 2000 and triplicate MS/MS scans of the three most abundant base peak (BP) ions, was employed. Equine skeletal muscle myoglobin (Sigma-Aldrich, M0630, St. Louis, Mo.) was analyzed as a sample preparation and instrument performance standard. The resulting MS and MS/MS data sets were processed using Bioworks© (Thermo Electron, Version 3.1) and Xcaliber© Software (Thermo Electron, Version 1.3). Except where noted, fragment ion identity assignments were based upon automated software MS/MS analysis of primary-ion peak fragments with software default Xcorr thresholds set for assignment acceptance. The sequence coverage for the mutant myoglobin standard was 100%.

### Reagent Scouting

For disulfide-linked dimer dispersal scouting, a sub-fraction of purified F1-V_{MN} formulated at ˜0.7 mg/ml in 20 mM L-arginine, 10 mM NaCl, pH 9.9, without added 1 mM L-cysteine, was air oxidized to form ˜22% disulfide-linked dimer. Reagents were added from un-adjusted, acidic, 100-mM stocks of freshly prepared DTE, L-cysteine, and IAA. For the two-reagent conditions, the reductant was added first, followed by a 10-min hold at 25° C. before adding IAA. Adjusted samples were held at 25° C. within the HPLC-SEC auto injector before analysis. Samples were analyzed through HPLC-SEC with two tandem columns (G3000SWxl) on an Agilent 1100 system (Agilent Technologies, Palo Alto, Calif.) eluted at 0.8 ml/min with 0.1 M KH₂PO₄, 0.1 M Na₂SO₄, 0.3 M NaCl, pH 7.0. Column performance was confirmed by running high MW size standards (BioRad). The percentage of integrated A₂₃₀ eluting in each peak relative to total protein-related integrated absorbance was calculated within Chemstation 2.0 software (Agilent).

For non-covalently-linked multimer dispersal scouting, reagents were prepared as 10-fold stocks in high-quality water and adjusted as needed to ˜pH 6.5. An aliquot of F1-V_{MN}, initially formulated at ˜0.7 mg/ml in 20 mM L-arginine, 10 mM NaCl, 1 mM L-cysteine, pH 9.9, was titrated by micro-addition of HCl to pH 6.5. In less than 5 minutes, the aliquots were divided and transferred with mixing into containers pre-loaded with 1/10^{th} volume of additive stocks. Samples were held at 4° C. before HPLC-SEC analysis by methods similar to those described for disulfide-linked dimer dispersal scouting above.

### Animal Vaccinations

Research was conducted in compliance with the Animal Welfare Act and other federal statutes and regulations relating to animals, and experiments involving animals were conducted according to the principles set forth in the Guide for the Care and Use of Laboratory Animals. The facility where this research was conducted is fully accredited by the Association for Assessment and Accreditation of Laboratory Animal Care International. Groups of 10 female, 8- to 10-week-old outbred (Hsd:ND4) Swiss Webster mice were inoculated subcutaneously (s.c.) with purified, recombinant F1-V_{STD}, F1-V_{MN}, F1-V_{AG} or F1-V_{C424S-MN} preparations. To evaluate the effect of aggregation state on the protective efficacy of F1-V as well as the efficacy of the new F1-V_{C424S}, various F1-V aggregation state formulations were produced. Vaccine candidate formulations included monodisperse F1-V_{C424S-MN}, the cysteine-capped, monodisperse F1-V_{MN}, and the converted multimer, F1-V_{AG}. F1-V_{AG} was produced by incubating F1-V_{MN} overnight at pH 5.1 and 4° C. to enhance F1-V aggregation. One group of 10 mice was inoculated s.c. with the previously reported mixed solution state F1-V_{Std} as a positive control (Powell et al., (2005) Biotechnol. Prog. 21 (2005), pp. 1490-1510). In order to maximize immunogenicity, each protein antigen was adsorbed to aluminum hydroxide adjuvant (Alhydrogel. 1.3%; Superfos Biosector, Vedbaek, Denmark; 0.19 mg of aluminum per dose), critically before exposure of adjuvant to injection buffer (1×PBS). Each antigen-adjuvant mixture (200 μL) containing 20 μg of each antigen was administered at a single subcutaneous site on the backs of the animals. After 30 days, the animals were boosted with an identical dose at the same injection site.

### Measurement of Serum Antibody Titer Using ELISA

Mice were anesthetized with a mixture of 5 mg of xylazine (XYLA-JECT; Phoenix Pharmaceutical, Inc., St. Joseph, Mo.) per kg, 0.83 mg of acetylpromazine (Fermenta Animal Health Co., Kansas City, Mo.) per kg, and 67 mg of ketamine hydrochloride (Ketamine; Phoenix Pharmaceutical, Inc.) per kg administered intramuscularly. Blood was collected by retro-orbital sinus puncture for the determination of antibody titers 56 days after the initial injection by standard enzyme-linked immunosorbent assay (ELISA). Briefly, 100 ng of each purified protein in carbonate buffer, pH 9.4, was applied to each well of a 96-well microtiter plate and allowed to incubate overnight at 4° C. Plates were then washed with 1×PBS+0.05% Tween 20. Plates were blocked with 100 μl of assay diluent (1×PBS, 1% bovine serum albumin, 0.05% Tween 20) for 1 hour at 37° C. Plates were washed again and serial dilutions of antiserum in assay diluent ranging from 1:50 to 1:2,048,000 were applied in triplicate. Plates were allowed to incubate at 37° C. for 1 hour, washed, and a 1:5000 dilution of horseradish peroxidase-conjugated goat anti-mouse IgG was applied for 1 hour at 37° C. Plates were washed and the chromogenic substrate 3,3′,5,5′ tetramethylbenzidine (TMB; BD Biosciences, Pharmingen, San Diego, Calif.) was added. After a 30-minute incubation at 37° C. in the dark, the reaction was stopped with 25 μl of 2 N sulfuric acid. Plates were read at an optical density of 450 nm (OD₄₅₀).

Y. pestis Lethal Challenge

Each of the vaccinated animals designated to receive s.c. challenges was administered 10⁴, 10⁷, 10⁸, or 10⁹ 50% lethal doses (LD₅₀) of wild-type Y. pestis CO92, 30 days after the booster dose. The s.c. LD₅₀ for adult mice challenged with CO92 is 1.9 colony-forming units (CFU) as determined by serial dilution and plating. The mice were observed daily for 28 days, at which time the survivors were killed. Fisher's two-tailed exact tests were used to evaluate animal survival data. Mean time to death after lethal plague challenge was evaluated using Student's t-tests. Significance in pair-wise comparisons of delayed time to death between groups was computed using Student's t-tests.

### Example 2

### F1-V_{MN} and F1-V_{C424S-MN} Expression

This Example demonstrates the expression of two F1-V fusion proteins, F1-V_{MN} and F1-V_{C424S-MN}. In order to evaluate the effect of super molecular structure (for instance, its state of aggregation) of the F1-V-based plague vaccine antigen on protective efficacy and to facilitate vaccine production, the sole cysteine (C424) in F1-V was replaced with a serine residue by site-directed mutagenesis. Standard F1-V_{MN} and the modified F1-V_{C424S-MN} proteins were independently over-expressed in E. coli, recovered by mechanical lysis/pH-modulation, and purified from urea-solubilized, soft inclusion bodies with successive ion-exchange, ceramic hydroxyapatite, and size-exclusion chromatography stages as described in Example 1. Aggregation characteristics for the purified proteins were characterized and compared under variable pH and buffer solution-additive conditions. The biological activities of the two purified proteins in various super molecular states were then evaluated for immunogenicity and efficacy in mice against lethal Y. pestis challenge.

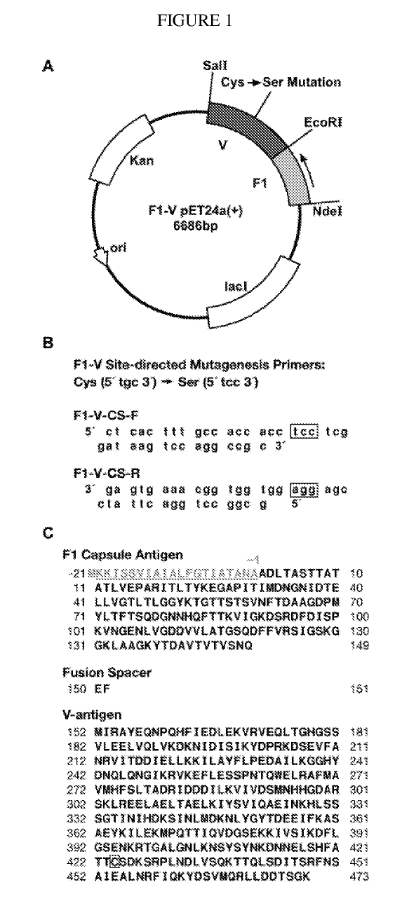

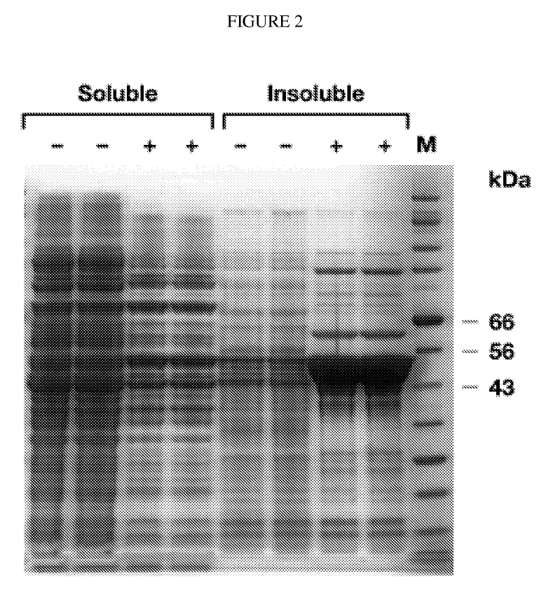

F1-V_{MN} and the modified F1-V_{C424S-MN} proteins were expressed as described in Example 1. The original pET-24a-based F1-V expression vector (pPW731) was modified by site-directed mutagenesis to replace the sole cysteine (Cys₄₂₄) with a serine residue (FIG. 1). This mutation was performed to eliminate the necessity for reducing conditions during the F1-V protein purification process and to evaluate the effect of the cysteine residue on F1-V protein aggregation. After induction with 0.5 mM IPTG, the F1-V_{C424S} vector over-expressed an insoluble 53-kDa protein as determined by SDS-PAGE (FIG. 2). A pH-based precipitation process was employed to enrich the F1-V_{C424S} protein before solubilization with 5M urea and ion-exchange chromatography.

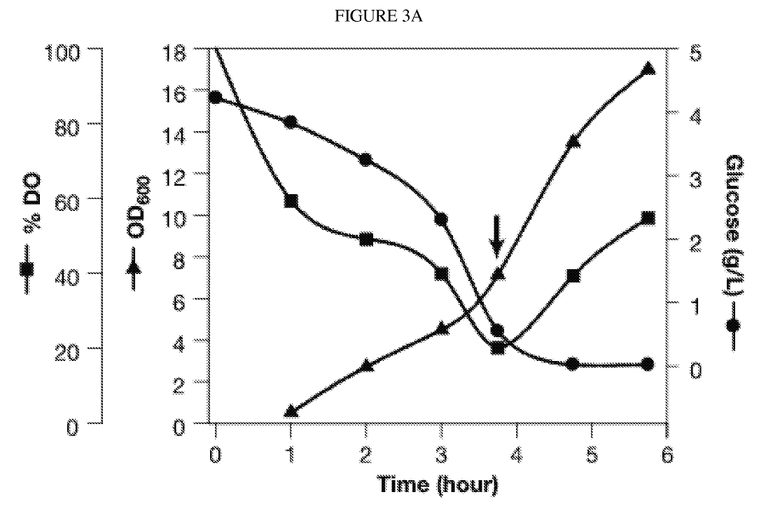

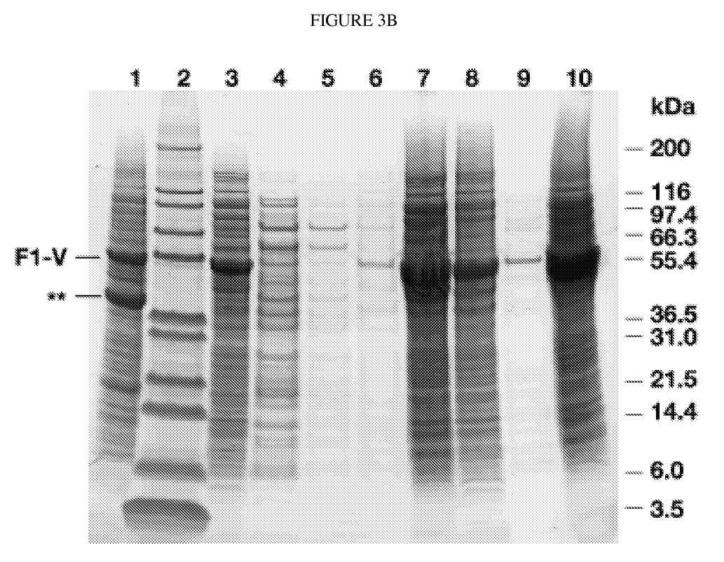

At the larger scale, the time course for cultivating E. coli., BLR130 transformed with pPW731 plasmid (containing the coding sequence for the unmodified, cysteine-containing F1-V (Powell et al., (2005) Biotechnol. Prog. 21 (2005), pp. 1490-1510)) had a controlled induction response (FIG. 3A). The resulting F1-V was precipitated by slow pH adjustment to pH 4.8 and additional contaminants were removed (compare lanes 1 vs. 7 in FIG. 3B) by pH modulation before re-solubilization of the enriched F1-V pellet in urea (FIG. 3B).

### Example 3

### Ion-Exchange Chromatography

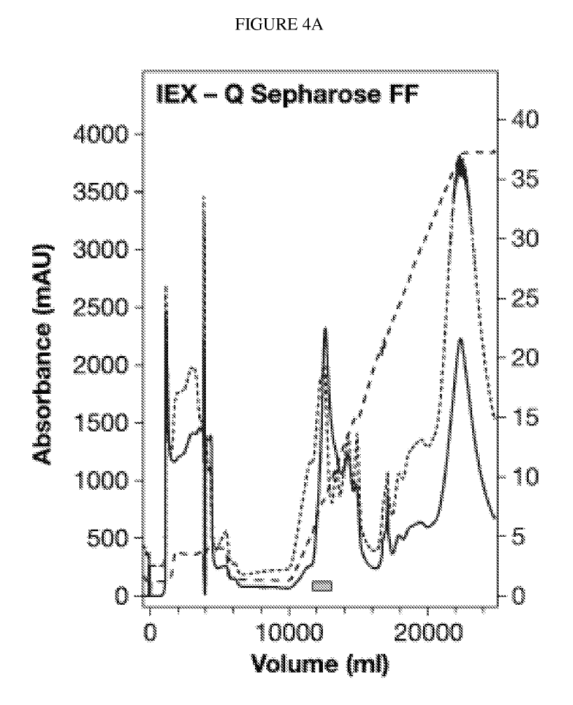

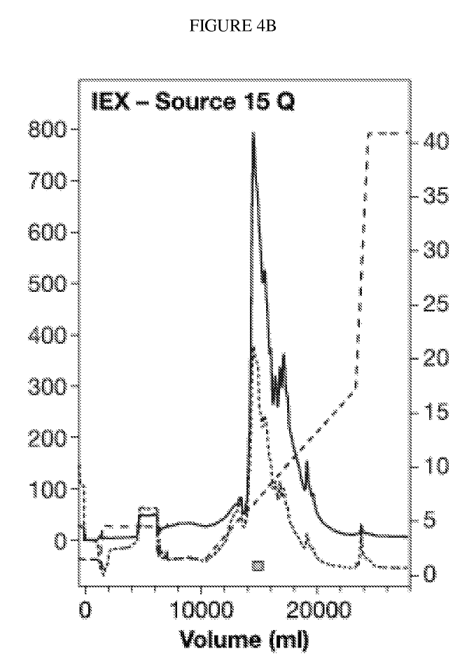

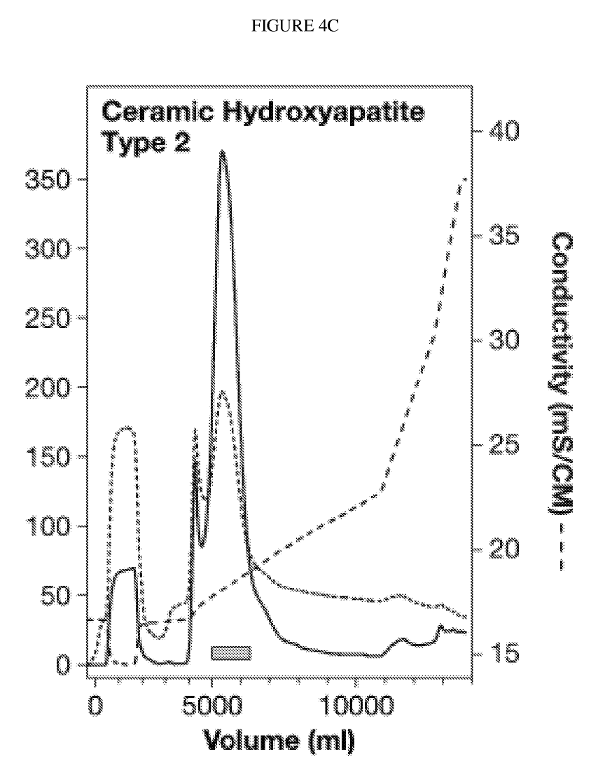

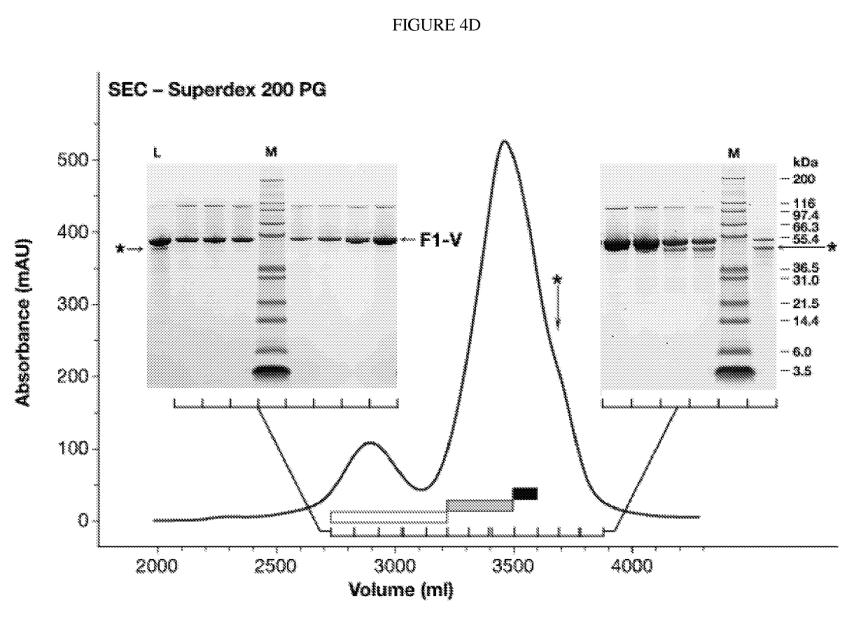

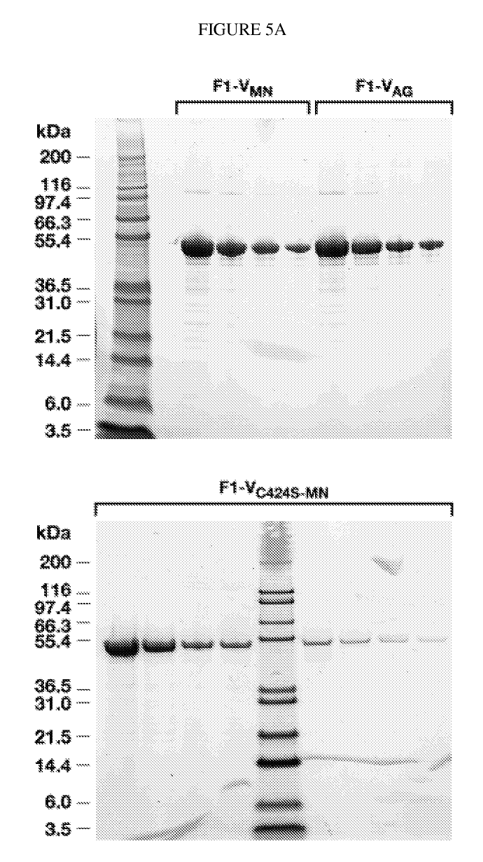

This Example demonstrates the purification of both F1-V and the variant F1-V_{C424S}. Standard F1-V and the F1-V_{C424S} variant purified similarly through the ion-exchange stages of the improved process. Profiles from purification of the standard (cysteine-containing) F1-V performed at the larger scale are shown (FIG. 4). The Q-Sepharose FF chromatography stage, performed under partially dissociating conditions and eluted with the denaturing Gdn cation, was effective as a charge-based step for isolating F1-V (FIG. 4A). F1-V_{C424S} monomer eluted at Gdn HCl concentrations below 100 mM, similar to what was observed with F1-V_{STD-MN}. Dimer and trimer forms of F1-V remained intact during reducing SDS-PAGE analysis when samples were prepared by heating to less than 70° C. for 10 minutes. These same forms were dispersed and ran as apparent F1-V monomers when heated to 100° C. for 10 minutes, corroborating prior observations of strong self association F1-V by gel electrophoresis (Powell et al., (2005) Biotechnol. Prog. 21 (2005), pp. 1490-1510). HPLC-SEC analysis confirmed that the trailing edge of the Q-Sepharose FF F1-V peak (elution volume 1400 ml, FIG. 4A) contained dimer and trimer forms of F1-V between 60 and 120 mM Gdn. A portion of F1-V remained in the Q-Sepharose FF non-bound fraction. Re-application to the hydroxide-stripped column recovered only a small proportion of the F1-V present, indicating the F1-V flow-through was not due to column overloading. This phenomenon also concurs with prior findings of an unrecoverable fraction consistently observed during F1-V purification (Powell et al., (2005) Biotechnol. Prog. 21 (2005), pp. 1490-1510). A subpopulation of F1-V within inclusions, relatively resistant to dissociation in 5 M urea, was likely excluded from the Q-Sepharose FF resin. The Source 15Q elution profile was characteristically jagged and extended with multiple, sharp, minor peaks apparently superimposed on top of a broader three-peak profile (FIG. 4B). The jagged nature of this elution profile was observed in multiple runs during development work and was not related to particular instrumentation or the range of the UV detector.

### Example 4

### Aggregate Dissociation

This Example demonstrates the conditions necessary to produce substantially monodisperse F1-V. To further elucidate F1-V losses to non-binding, small-scale F1-V multimer dissociation studies, monitored using HPLC-SEC analysis, showed that F1-V multimers were not fully dispersed by even 7 M urea. Maximum dispersal was observed using 6 M Gdn HCl. Upon buffer exchange over G-25 resin, from 6M Gdn HCl into Source 15Q Buffer, F1-V remained substantially monodisperse.

### Example 5

### Affinity Chromatography

This Example demonstrates the effects of using ceramic hydroxyapatite chromatography to further purify Fa-V and to exchange F1-V into non-denaturing conditions. Ceramic hydroxyapatite (CHT) chromatography, being insensitive to high concentrations of Gdn HCl, was used to exchange F1-V into non-denaturing conditions while providing additional purification. Including trace PO₄²⁻ and Ca²⁺ ions was critical for efficient F1-V binding and resin stability. Predominantly lower molecular weight contaminants flowed through the CHT-T2 stage. F1-V, processed over CHT Type 2 (T2) resin, eluted primarily in monomeric form (>80%), free of denaturing agents (FIG. 4C). F1-V_{C424S} recovered from the CHT Type 1 (T1) resin contained higher levels of dimer, trimer, and multimer (˜73%). The prior reported method similarly removed denaturants while F1-V was bound to ion-exchange resin (Powell et al., (2005) Biotechnol. Prog. 21 (2005), pp. 1490-1510).

### Example 6

### SEC (Size-Exclusion Chromatography)

This Example describes SEC purification of F1-V_{(MN)} and F1-V_{C424S}. Superdex 200 PG SEC provided a convenient method for combined final formulation and size classification. F1-V_{(MN)} and F1-V_{C424S} purified similarly by SEC. The mobile phase containing physiologically compatible additives, L-arginine for buffering at pH 10.0, and L-cysteine for thiol capping, maximized the monodispersity of F1-V. Low molecular mass protein trace contaminants in the range of 40 to 49-kDa overlapped with the monomer peak trailing edge (FIG. 4D, asterix and black bar). The major contaminant at ˜49-kDa was identified by N-terminal sequencing as E. coli serine hydroxymethyl transferase. Separating and selectively pooling the purest fractions based upon SDS-PAGE analysis minimized these trace contaminants (FIG. 4D, the three rightmost product lanes were not pooled). Although not used for the vaccination trials, the dimer/multimer pool was essentially 100% pure F1-V with no detectable low molecular mass contaminants by SDS-PAGE (FIG. 4D, Lanes 2, 3, 4, and 6 from the left). Thus, after initial purification, F1-V and F1-V_{C424S} preferentially self-associated while the 40- and 49-kDa trace contaminants remained as apparently low molecular species. The monomeric and dimeric F1-V forms were well-separated, especially when tandem columns were employed. Thus, the use of a size-based purification method as the last stage critically ensured maximally monodisperse F1-V for use in vaccination trials.

### Example 7

### Protein Purification Process Yield

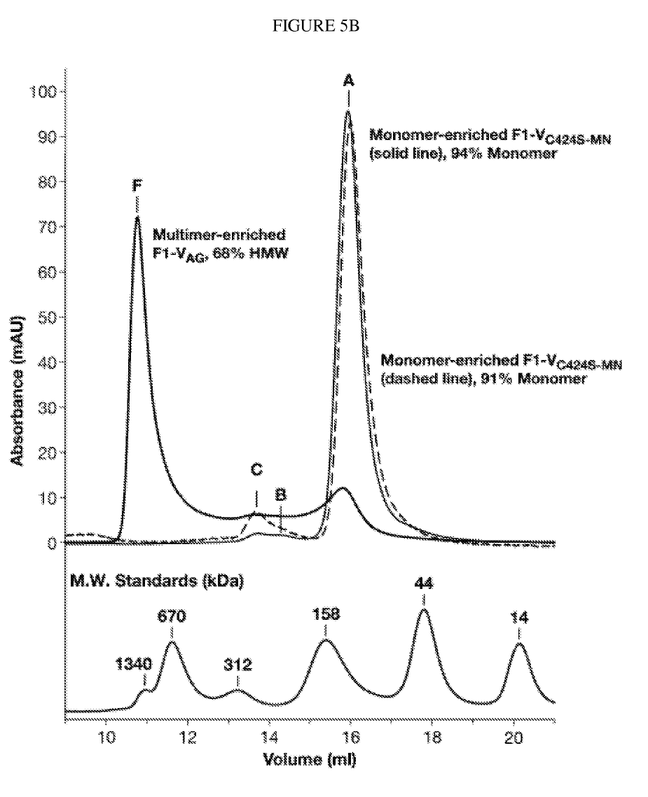

This Example describes the protein purification process yield with F1-V and F1-V_{C424S-MN}. From 765 g of cell paste, 823 mg of monodisperse F1-V was recovered for a final process yield of ˜1.2 mg/g of cell paste (Table 1A). From 23.2 g of F1-V_{C424S} cell paste, 40 mg of F1-V_{C424S-MN} was recovered for a process yield of ˜2 mg/g of cell paste (Table 1B). Purity, identity and protective potency testing reported herein were conducted on intermediate bulk materials, prior to final finishing. SDS-PAGE and HPLC-SEC profiles of purified F1-V_{MN}, F1-V_{AG}, and F1-V_{C424S-MN} confirmed greater than 95% purity for the preparations (FIGS. 5A and 5B). Each preparation was specifically detected in immunoblot analysis as per previously reported methods (Powell et al., (2005) Biotechnol. Prog. 21 (2005), pp. 1490-1510) versus mouse anti-F1 and anti-V antibodies. Low endotoxin levels (<0.5 EU/mg) and host cell genomic DNA levels (<2 pg/mg) were typically observed.

TABLE 1A
F1V
Process Summary for F1-V_{MN}

| | Concentration | Volume | Total Protein | Step Yield | Yield (mg TP |
|---|---|---|---|---|---|
| Production Stage | (mg/mL) | (mL) | by A280 (mg) | (%) | per g CP) |
| Fermentation (20L) | — | — | — | — | (765 g CP) |
| Wet Cell Paste (CP) | | | | | |
| Solubilized Pellet | 27.0* | 3,300 | 89,100* | — | 117 |
| Ion Exchange, | 12.6 | 1,700 | 21,500 | 24 | 28 |
| Q-Sepharose FF | | | | | |
| Ion Exchange, | 5.1 | 1,000 | 5,100 | 24 | 6.6 |
| Source 15Q | | | | | |
| Affinity, Ceramic Hydroxyapatite | 7.4 | 285 | 2,115 | 42 | 2.8 |
| Type 2 & Concentration | | | | | |
| Size Exclusion, | 0.78 | 1,055 | 823 | 39 | 1.1 |
| Superdex 200 PG | | | | | |

*Estimated A₂₈₀ with E = 1.0.

TABLE 1B
Process Summary for F1-V_{C424S-MN}

| | Concentration | Volume | Total Protein | Step Yield | Yield (mg TP |
|---|---|---|---|---|---|
| Production Stage | (mg/mL) | (mL) | by A280 (mg) | (%) | per g CP) |
| Fermentation (10 L) | — | — | — | — | (23 g CP) |
| Wet Cell Paste (CP) | | | | | |
| Solubilized Pellet | *110 | 35 | 3,850* | — | 167 |
| Ion Exchange, | 3.5 | 140 | 490 | 13 | 21 |
| Q-Sepharose FF | | | | | |
| Ion Exchange, | 3.2 | 80 | 256 | 52 | 11 |
| Source 15Q | | | | | |
| Affinity, Ceramic Hydroxyapatite | 5 | 47 | 235 | 92 | 10 |
| Type 1 & Concentration | | | | | |
| Size Exclusion, | 0.77 | 52 | 40 | 20 | 1.7 |
| Superdex 200 PG | | | | | |
| & Concentration | | | | | |

*Estimated A₂₈₀ with E = 1.0

### Example 8

### Solution Stability Versus pH

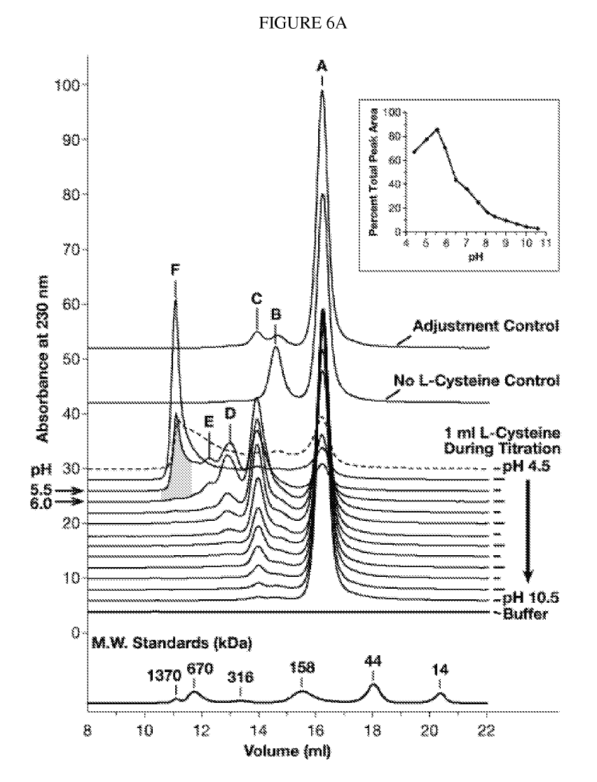

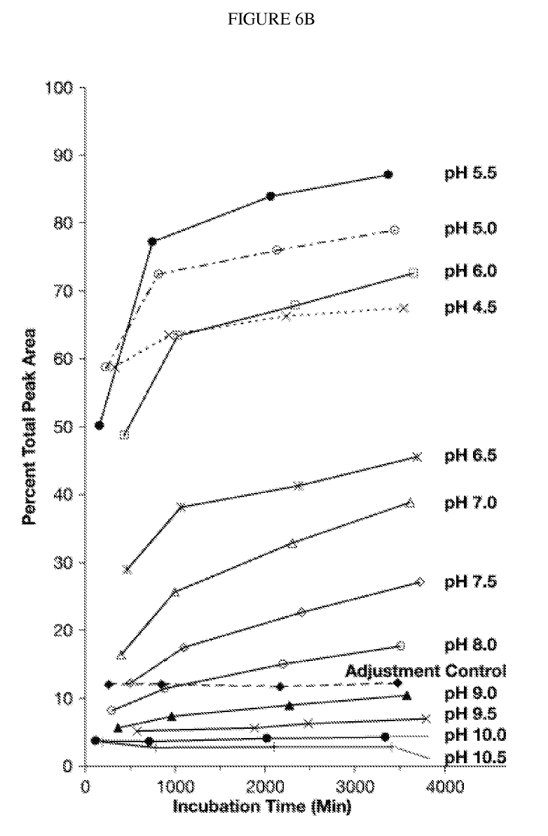

This Example demonstrates the effect of pH on aggregation of the F1-V fusion protein. Although the handling of F1-V under neutral to acidic conditions was previously known to be problematic, the details of such effects were not described (Powell et al., (2005) Biotechnol. Prog. 21 (2005), pp. 1490-1510). As part of an effort to stabilize the monomeric state of F1-V preparations, the solution structure of F1-V was further characterized as a function of diluent pH. Analytical size-exclusion chromatography over a silica-based, wide-pore G3000SWxl column was used to measure the effect of solution composition on the ratio of monomer to dimer/trimer/multimer species as well as the effect on F1-V_{(NC)} and F1-V_{(S—S)} dimer sub-classes. The unique F1-V_{C424S} form permitted separate assessments of the effects of reducing agents and stabilizing additives on structure. A clear trend toward formation of higher molecular mass F1-V associations was observed as a function of lowering solution acidic pH (FIG. 6A). An identical apparent size profile versus pH trend was observed for F1-V_{C424S-MN} in formulation buffer lacking L-cysteine except that the shoulder corresponding to disulfide-linked F1-V dimer (FIG. 6A, Peak B) was no longer a observed as distinct feature. As shown in the inset to Panel A, the greatest percentage of high molecular mass species appeared between pH 6 and 8. Additionally, the percentage of very high molecular mass species increased as the solution pH dropped from pH 6.0 to 5.5 (FIG. 6, shaded area, Peak F). Aggregation was further exacerbated as solution pH dropped below pH 5.5, observed as a loss of total protein from solution (FIG. 6A, Inset).

For proteins, the histidine imidazole and the N-terminal amine groups become positively charged and thereby decrease the net protein negative charge (calculated F1-V pI=5.19) below pH 8.0. Without being bound by theory, it is likely that F1-V multimerization at low pH involves the loss of ionic repulsive forces. A structural re-arrangement exposing hydrophobic patches is also consistent with the pH trend data. The conversion to multimer was time-dependent as shown by the limited conversion to multimer observed in the “adjustment control” sample that was titrated from pH 4.5 back up to pH 9.9 within 10 minutes of acidification. This time dependence was also clear after plotting the percentage of high molecular mass species versus hold time for each pH condition (FIG. 6B) where transitions to the stable profiles shown in Panel A were quite slow. This would also be consistent with a relatively slower structural re-arrangement during F1-V multimer formation. These results demonstrate that, in the presence of optimized solution additives, moderately basic pH conditions were critical to maintaining a monodisperse F1-V preparation. Thus, preparation under basic conditions and formulation at pH 9.9 maximized recovery of monomer. This concurs with prior empirical findings of optimal F1-V purification at pH 9.5 (Powell et al., (2005) Biotechnol. Prog. 21 (2005), pp. 1490-1510).

### Example 9

### Additive Study

### Thiol Reducing and Blocking Agents

This Example demonstrates the effect of formulation additives intended to minimize disulfide-linkage. The ability of HPLC-SEC analysis to separate disulfide-bonded F1-V_{(S—S)} dimer (FIG. 6A; Peak B) from non-covalently associated F1-V dimer, (Peak C) enabled the evaluation of formulation additives intended to minimize disulfide-linkage (Table 2). High DTT concentrations (10 mM) resulted in complete disulfide bond disruption for >20 hours at 25° C. Trace DTT concentrations (0.5 and 1.0 mM) resulted in initial disruption followed by reformation of 35% of F1-V_{(S—S)} dimer after 10 hours. Intriguingly, the F1-V_{(S—S)} percentage decreased to starting dimer levels after a further hold for 10 hours. This observation did not fit a simple disulfide bond exchange/oxidative disulfide bond formation model, and, without being bound by theory, may have been an example of further oxidation (Huxtable, Biochemistry of the Sulfur, Plenum Press, New York, 1986, pp. 207-208). The addition of iodoacetamide alone (2.5 mM), an irreversible free-thiol capping reagent, led to a minor decrease in the F1-V_{(S—S)} dimer over ˜22 hours. However, when the F1-V_{(S—S)} dimer was first reduced with DTT (0.5 or 1 mM), held for 10 minutes at 25° C., and the resulting free-thiols blocked by excess iodoacetamide (1.25 or 2.5 mM), the level of F1-V_{(S—S)} remained below 5% for more than 20 hours.

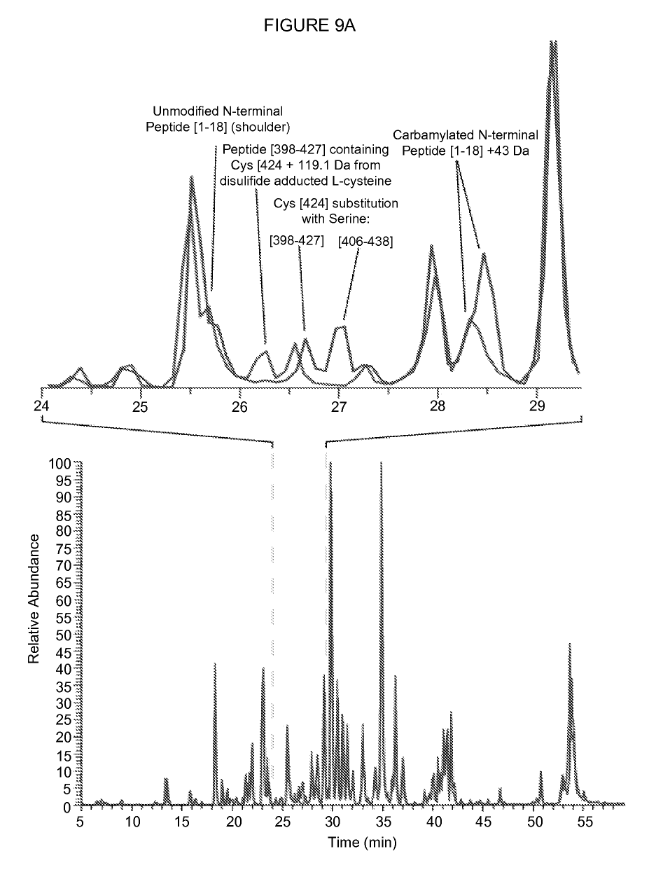

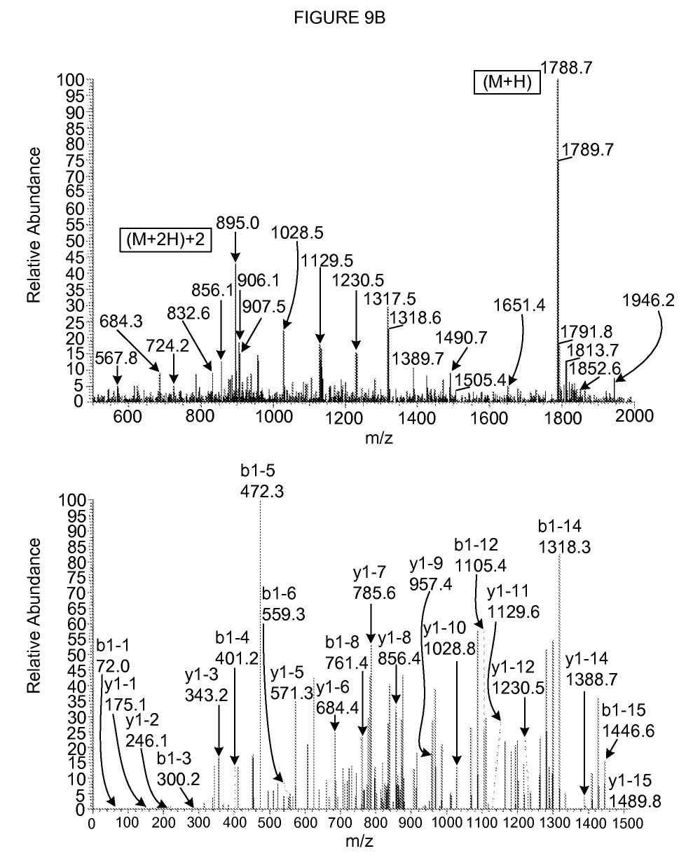

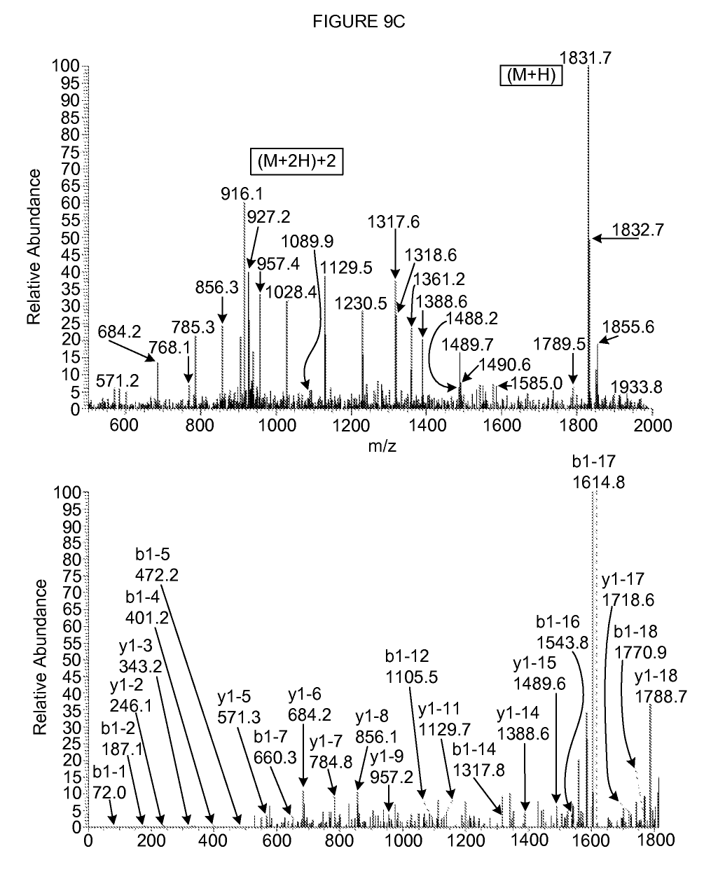

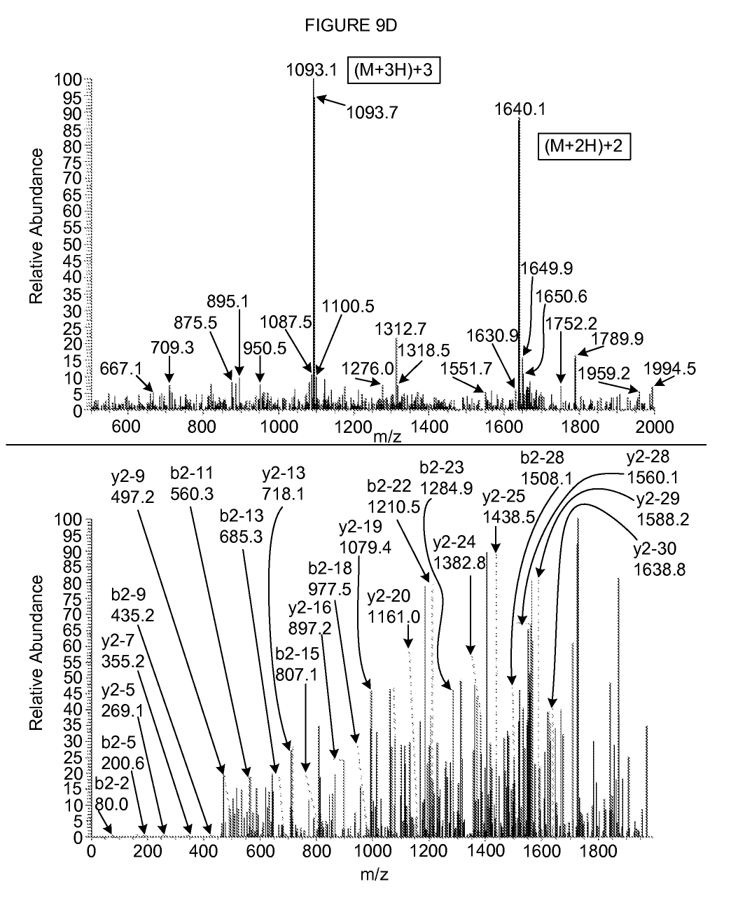

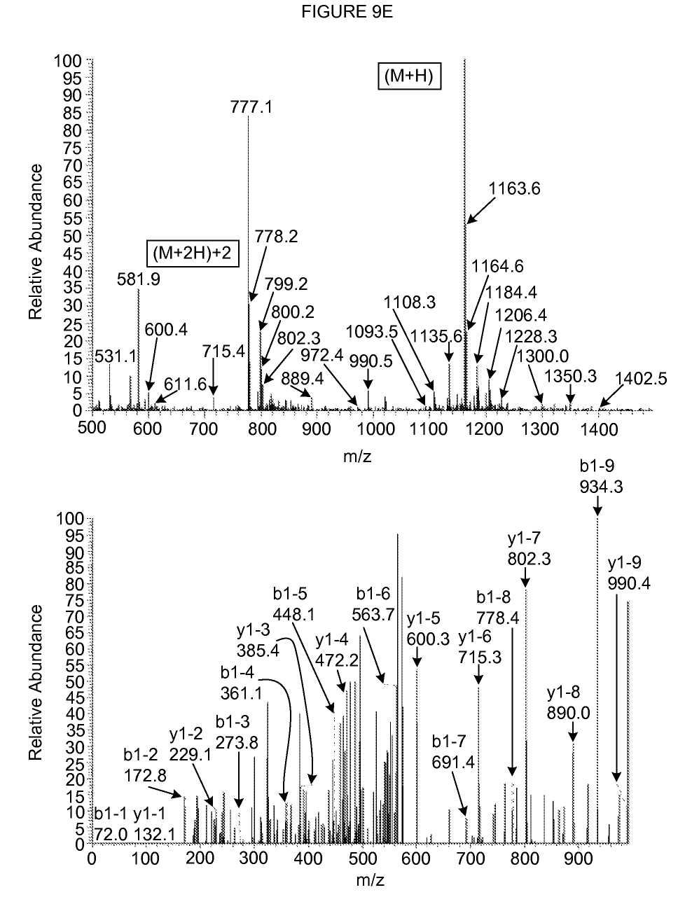

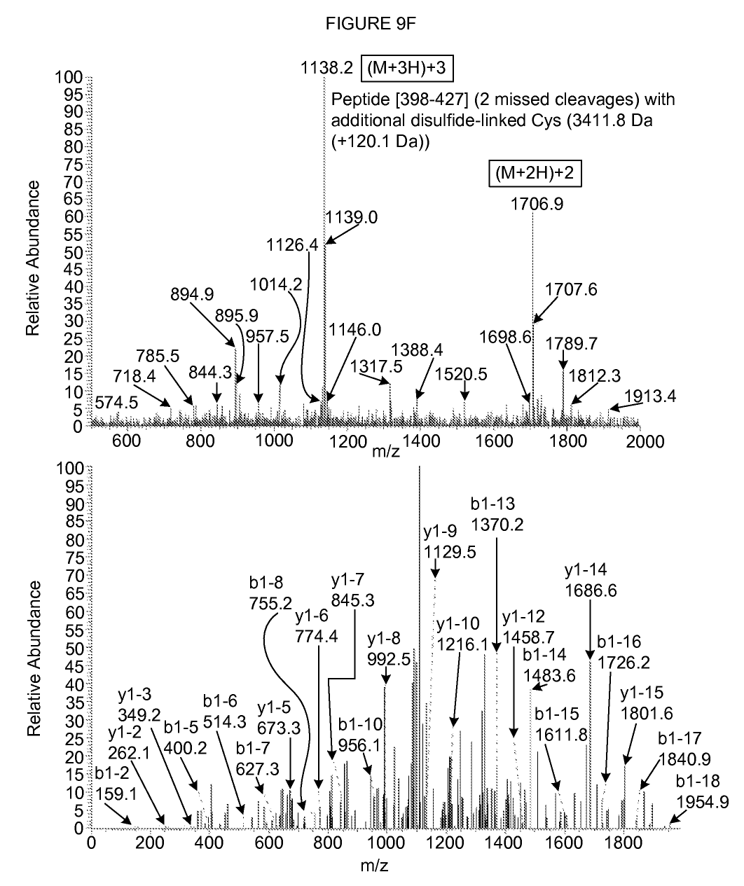

Unexpectedly, exposing F1-V_{(S—S)} to trace levels of L-cysteine (0.5 mM or 1 mM) alone provided durable disulfide bond disruption. L-cysteine was superior to other additives studied for disulfide disruption, showing an essentially invariant 3.3% F1-V_{(S—S)} content after 20 hours at 25° C. Without being bound by theory, L-cysteine may have formed a relatively more stabile adduct to the F1-V free-thiol than other reducing agents, perhaps stabilized by local ionic or hydrogen bond interactions. This concept was supported by mass spectrum analysis of tryptic peptides where the uncapped, free-cysteine-containing fragment was not recovered but the cysteine-adduct fragment was isolated and identified with high confidence by MS/MS fragmentation analysis (FIG. 9). As a non-toxic, physiological amino acid, L-cysteine (1 mM) was subsequently selected as the agent of choice for suppressing F1-V_{(S—S)} disulfide-bonded dimer levels in native F1-V_{MN} and F1-V_{AG} formulation buffers.

TABLE 2

| | Disulfide Linked Dimer | | | | | |
|---|---|---|---|---|---|---|
| Additive Condition | min | % | min | % | min | % |
| No Additions | 68 | 21.4 | 522 | 22.8 | 975 | 23.1 |
| 10 nM DTE | 417 | 3.1 | 871 | 3.5 | 1385 | 4.0 |
| 0.5 mM DTE | 242 | 30.7 | 696 | 35.8 | 1150 | 23.7 |
| 1 mM DTE | 103 | 3.8 | 556 | 36.8 | 1010 | 21.1 |
| 2.5 mM IAA | 382 | 15.7 | 836 | 13.2 | 1290 | 10.8 |
| 0.5 mM DTE, 1.3 mM IAA | 312 | 3.3 | 766 | 3.9 | 1220 | 3.7 |
| 1 mM DTE, 2.5 mM IAA | 173 | 2.6 | 626 | 3.8 | 1080 | 4.1 |
| 0.5 mM L-Cys, 1.3 mM IAA | 347 | 4.1 | 801 | 3.7 | 1255 | 3.8 |
| 1 mM L-Cys, 2.5 mM IAA | 208 | 3.8 | 661 | 4.8 | 1115 | 4.4 |
| 0.5 mM L-Cysteine | 277 | 2.8 | 731 | 3.7 | 1185 | 3.3 |
| 1 mM L-Cysteine | 138 | 3.0 | 591 | 3.5 | 1045 | 3.3 |

### Example 10

### Non-Covalent Multimer Modulation

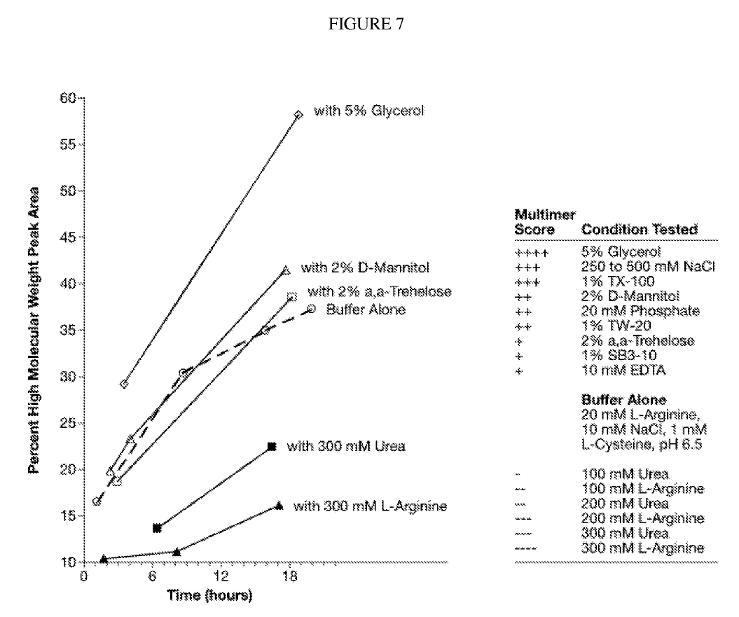

This Example demonstrates the effect of solution additives on F1-V monodisperse solution stability under conditions of disulfide-bond suppression (1 mM L-cysteine) and non-covalent multimer potentiation (pH 6.5; FIG. 7). Several common formulation additives promoted F1-V self-association, including glycerol, non-reducing sugars, common buffer salts, a non-ionic detergent and a zwitterionic detergent. The multimer-inducing effect of glycerol was surprising in light of its common use to stabilize proteins in solution. These results indicate the existence of a strong, non-covalent (for instance, non-sulfhydryl) self-binding energy within the F1-V protein that drives self-association. Urea and L-arginine suppressed multimer formation with L-arginine (0.3 M) being the most effective F1-V multimer-suppression additive examined. In separate survey, conducted at pH 10.0, L-arginine was more effective than L-lysine for F1-V monomer stabilization. Thus, the L-arginine guanidinium group may be key to F1-V monomer stabilization. These additive trends informed manufacturing process design and monodisperse F1-V final formulation.

### Example 11

### Freeze Drying Survey

This Example demonstrates lyophilization of F1-V. The materials used in the vaccination protocol (Examples 14, 15) were not lyophilized. Anticipating the eventual need for a chilled storage product form, lyophilization of F1-V was evaluated. Disulfide-linked □immer formation (˜35% □immer, no trimer) was observed in F1-V samples lyophilized without L-cysteine in the formulation buffer (in 20 mM L-arginine, 10 mM NaCl with 2% D-mannitol, pH 9.9) and reconstituted with water. This emergent □immer was dispersed by reconstitution with added 1 mM L-cysteine yielding ˜1.7% non-covalent □immer and ˜2.0% disulfide-linked □immer. Minimal F1-V non-covalent □immer formation was observed after lyophilization in formulation buffer supplemented with 2% D-mannitol. This contrasted with the destabilizing effect of 2% D-mannitol observed at pH 6.5 (FIG. 7). Lyophilization with added 1 mM L-cysteine resulted in no discernable increase in □immer content upon rehydration relative to the pre-lyophilization material. Thus, it can be practical to store F1-V prepared lyophilized in 20 mM L-arginine, 10 mM NaCl, 1 mM L-cysteine, 2% D-mannitol, pH9.9.

### Example 12

### Peptide Mapping

This Example demonstrates peptide mapping of the F1-V and F1-V_{C424S} fusion proteins. F1-V and F1-V_{C424S} identities were determined by peptide mapping (FIG. 9). The tryptic-digest sequence coverage for F1-V and F1-V_{C424S} were 73.0 and 85.3%; and for chymotrypic-digest, 58.0 and 61.8%, respectively-confirming target protein expression and recovery. The F1-V tryptic (M+H=1788.7 Da) and chymotryptic N-terminal peptides were positively identified, and supported the des-Met form of F1-V as reported previously (Powell et al., (2005) Biotechnol. Prog. 21 (2005), pp. 1490-1510). A modified N-terminus tryptic peptide (M+H=1831.7 Da, +43.0 Da) was identified. Using high-stringency fragment ion identification criteria (±0.2 Da, Xcorr≧1.5), searching the centriod data set for carbamylated N-terminal MS/MS ions (+43.0058) identified 4 b-ion identifications versus only 2 b-ion identifications for an acetylation (+42.0105) hypothesis. Thus, based upon parent and MS/MS ion identifications, N-terminal carbamylation was most strongly supported by the data. Base-peak profiles containing peaks for both the native and modified N-terminus indicated that the proportion of modification was slightly elevated in the F1-V_{C424S} preparation.

The serine mutation in F1-V_{C424S} was confirmed by identification of two tryptic peptides containing serine 424 (residues 398-427, M+2H+2=1640.1 Da and residues 406-438, M+2H+2=1881.4 Da), and of a single chymotryptic peptide (residues 421-431, M+H=1162.5 Da). The corresponding peptides were not found within the F1-V tryptic or chymotryptic MS data sets, confirming assay specificity.

Using automated methods, the F1-V peptides containing cysteine 424 were not identified in tryptic or chymotryptic digests. By visual inspection, a single peak unique to the F1-V tryptic-digest (FIG. 9 at ˜26.2 minutes within the base-peak profile overlay) remained unassigned. The major ion within this peak corresponded to a 3,411.8 Da peptide that matched the predicted molecular mass for residues 398-427 (3,292.5 Da) if one assumed cysteine 424 was covalently linked to free L-cysteine from the formulation buffer (molecular mass=121.1 Da, minus 2H lost upon formation of the disulfide bond). Subsequent examination of MS/MS the fragmentation pattern for this peptide confirmed this assignment. The corresponding peptide was not found within the F1-V_{C424S} tryptic MS data set, further demonstrating assay specificity. Thus, the identities of the native F1-V and F1-V_{C424S} genetic mutant preparations were positively confirmed.

### Example 13

### SEC-MALLS

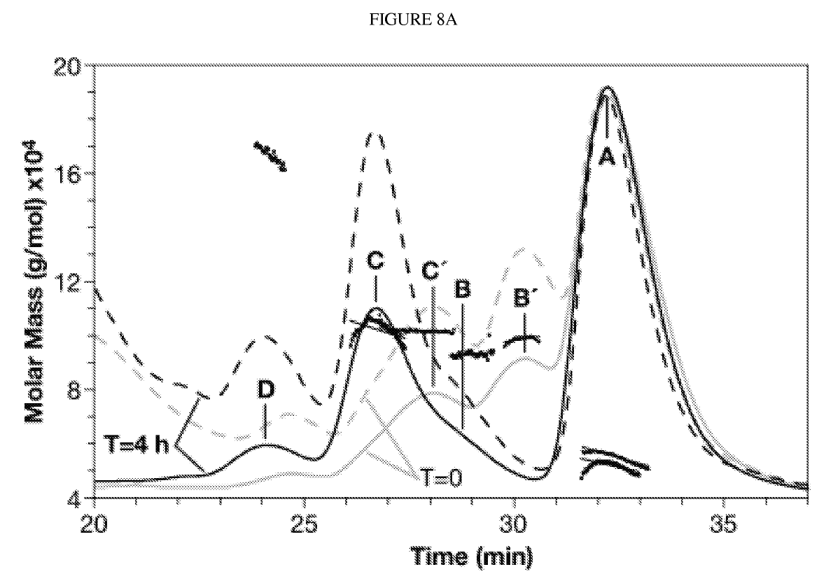

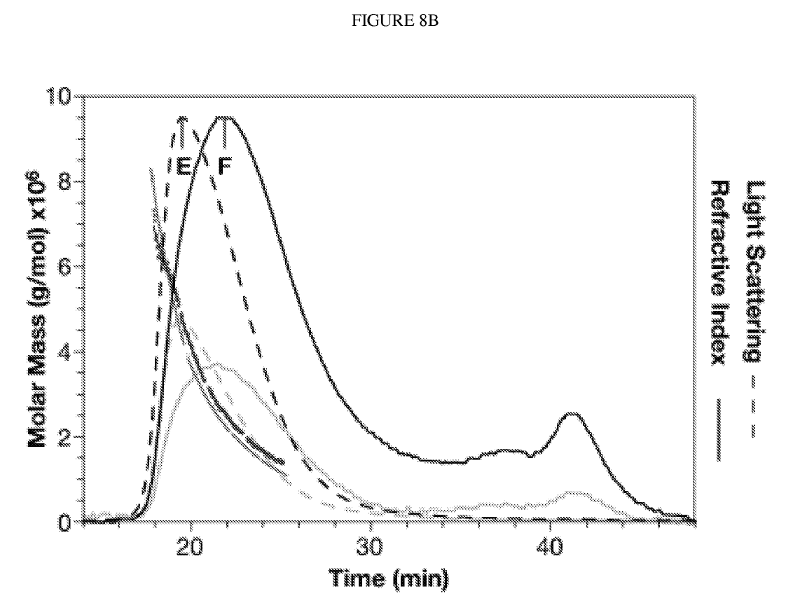

This Example demonstrates confirmation by SEC-MALLS that adjustment to pH 5.0 induced formation of an extensively multimerized F1-V population. Multiple-angle laser light scattering analysis was performed to assign F1-V solution states to HPLC-SEC assay elution profiles. Based on HPLC-SEC retention volumes alone, the major F1-V peak would have been assigned a molecular weight of ˜100 kDa relative to BioRad high MW size standards (FIG. 6, pH 10 Trace, Peak A). However, by SEC-MALLS the major peak was determined to have an absolute molecular mass between 52.0-55.2 kDa that closely matched the 54 kDa molecular weight expected for F1-V monomer (FIG. 8, Peaks A′ and A). Peak A was thus assigned as monomeric F1-V. This illustrated the known advantage of SEC-MALLS over the conventional methods using reference standards, as SEC protein elution times are known to be affected by differences in molecular radii, molecular shape, and affinities for the column packing.

Upon addition of 1 mM L-cysteine to and adjustment of monomeric F1-V to pH 6.5, a complex transition was observed wherein dimeric F1-V species formed at T=0 (FIG. 8A, Peaks B′-98.5 kDa and C′-101.8 kDa) and, with time, converted into earlier eluting, apparently more extended, dimeric species (FIG. 8A, Peaks B-93.1 and C-102.2 kDa). Based upon HPLC-SEC data alone, the ‘F1-V final □immer’ would have been incorrectly assigned as a tetramer (FIG. 6A, Peak C). Similarly, a well-separated peak with absolute molecular mass of ˜167 kDa was assigned as trimeric F1-V (FIG. 8A, Peak D). Thus, SEC-MALLS analysis permitted the unequivocal calibration of the SEC-HPLC elution profile for use in establishing that monomeric (monodisperse) preparations had been isolated.

After incubation of F1-V monomer at pH 5.0, SEC-MALLS analysis showed conversion to very high molecular mass solution states extending above 1 Mda, with data going off-scale at the beginning of the void peak (FIG. 8B, Peaks E and F). Thus, SEC-MALLS confirmed that adjustment to pH 5.0 induced formation of an extensively multimerized F1-V population.

### Example 14

### ELISA Response to F1-V Vaccinations

This Example demonstrates the ELISA response against F1-V vaccinations. ELISA was performed to determine the anti-F1 and anti-V IgG antibody response against F1-V_{AG}, F1-V_{C424S}, F1-V_{STD}, and F1-V_{MN} (Table 3). As previously observed (Heath et al., (1998) Vaccine 16, pp. 1131-1137; Powell et al., (2005) Biotechnol. Prog. 21, pp. 1490-1510) the IgG response was dramatically higher against the V antigen compared to the F1 protein for all of the F1-V fusion constructs (Table 3). The average geometric mean anti-V antibody titer was greatest against F1-V_{C424S} but not statistically different than that observed for prior standard preparations of F1-V_{STD} (119,000 versus 62,000, with a sample size of 30 mice per group). Anti-V antibody titers were statistically equivalent for all of the evaluated F1-V formulations, suggesting that the modified F1-V_{C424S} retains the capacity for recognition by protective anti-V antibodies. Thus, as there is no statistical difference between the anti-F1 and anti-V titers among these antigen groups, these findings indicate that F1-V aggregation state does not influence the capacity for protective antibodies to recognize the individual component proteins within the F1-V fusion protein.

The anti-F1 titers were substantially lower than anti-V titers for all fusion protein formulations, and these titers did not vary as much as the anti-V titers between the various treatments. A slightly lower, but not statistically significant, anti-F1 titer of 19,000 was observed for the positive control F1-V_{STD}, while the three additional F1-V formulations demonstrated identical average anti-F1 titers of 30,000. The F1 portion of F1-V was smaller and exhibited a less complex secondary structure than the V protein. Thus, it is not surprising to see less immunogenicity of F1, even after manipulation of the V antigen component.

TABLE 3

| Titer | | | Geometric | Lower | Upper |
|---|---|---|---|---|---|
| Type | Treatment | N | Mean | 95% CL | 95% CL |
| V | ALH | 10 | 300 | 300 | 300 |
| | F1-V_{AG} | 30 | 76,000 | 49,000 | 119,000 |
| | F1-V_{C424S-MN} | 30 | 119,000 | 79,000 | 179,000 |
| | F1-V_{STD} | 30 | 62,000 | 43,000 | 89,000 |
| | F1-V_{MN} | 29 | 86,000 | 54,000 | 136,000 |
| F1 | ALH | 10 | 300 | 300 | 300 |
| | F1-V_{AG} | 30 | 30,000 | 17,000 | 51,000 |
| | F1-V_{C424S-MN} | 30 | 30,000 | 17,000 | 54,000 |
| | F1-V_{STD} | 30 | 19,000 | 12,000 | 30,000 |
| | F1-V_{MN} | 29 | 30,000 | 16,000 | 55,000 |

### Example 15

### Protective Efficacy and Statistical Analysis

This Example demonstrates the protective efficacy of the various F1-V formulations. Purified F1-V formulations (F1-V_{MN}, F1-V_{AG}, F1-V_{C424S}, and F1-V_{STD}) were adsorbed to Alhydrogel (ALH) adjuvant in water, diluted into 1×PBS, and used to inoculate mice before s.c. challenge with 107-109 LD₅₀ of Y. pestis CO92. The Y. pestis CO92 strain is highly virulent as indicated by 100% fatality among ALH only-vaccinated mice at a much lower challenge dose (10⁴ LD₅₀ compared to 10⁷-10⁹ LD₅₀). All of the ALH control animals were dead by day 5 after challenge with an average time to death of 3.2 days. As indicated in Table 4, 100% of F1-V_{C424S} vaccinated mice survived lethal plague challenge with either 10⁷ or 10⁸ LD₅₀ Y. pestis CO92. In comparison, 70% of F1-V_{STD} animals survived challenge with either 10⁷ or 10⁸ LD₅₀ Y. pestis. Forced monomeric (F1-V_{MN}) and forced multimeric (F1-V_{AG}) forms of F1-V elicited 70-80% survival under the same challenge conditions. The protective efficacy of these F1-V-based vaccines was further demonstrated by 30-50% survival of mice when challenged with 10⁹ LD₅₀ Y. pestis.

Pairwise statistical comparisons were performed for all treatment groups. The statistical results indicate significant differences in “Percent Survival” among the various vaccination groups compared to the ALH control group (Table 4 Panel B). Statistically significant differences in survival were observed for all vaccination groups compared to the ALH control group at the 10⁷-10⁸ LD₅₀ dose range. Only F1-V_{STD} and F1-V_{MN} retained significant survival percentages at 10⁹ LD₅₀. Statistically significant differences in survival were not observed between the various vaccination treatments when compared to each other.

Whether or not those mice vaccinated with a given F1-V preparation, that died, survived longer than the control mice or mice vaccinated with another F1-V preparation, that died, is illustrated in Table 4, Panel B. The statistical comparison designated “Time to Death” highlights significant increases in average time to death among vaccinated mice compared to ALH-only control mice and to other vaccinated mice groups. For example, at 10⁷ LD₅₀, the average time to death for F1-V_{MN} vaccinated mice was 6.5 days, compared to 3.2 days for ALH inoculated mice. The difference in time to death between F1-V_{MN} and ALH groups was statistically significant (p<0.0032). F1-V_{C424S} statistical comparisons were not performed at the 10⁷ and 10⁸ challenge dose because none of the mice died under those conditions. The analysis indicates that all of the F1-V_{STD} vaccinated mice that died during the experiment, regardless of the challenge dose, lived significantly longer than the ALH control mice. Most of the other vaccinated mice (F1-V_{MN}/F1-V_{AG}/F1-V_{C424S}) that died, also survived significantly longer than the control mice. Significant differences in survival time between the test groups compared to each other were observed sporadically.

TABLE 4
Vaccinated Mouse Survival Data

| | | | | | | | Mean | Mean | | |
|---|---|---|---|---|---|---|---|---|---|---|
| | | Challenge | | | | Percent | Survival | Days to | | |
| Group | Treatment | Dose | Alive | Dead | Total | Survival | Time (SE) | Death (SD) | Min | Max |
| 1 | ALH | 10⁶ | 0 | 10 | 10 | 0 | 3.2 (0.3) | 3.2 (0.8) | 2 | 5 |
| 2 | F1-V_{STD} | 10⁷ | 7 | 3 | 10 | 70 | 21.7 (3.7) | 7.0 (0.0) | 7 | 7 |
| 3 | F1-V_{MN} | 10⁷ | 7 | 2 | 9 | 78 | 23.2 (4.2) | 6.5 (2.1) | 5 | 8 |
| 4 | F1-V_{AG} | 10⁷ | 8 | 2 | 10 | 80 | 23.2 (4.3) | 4.0 (1.4) | 3 | 5 |
| 5 | F1-V_{C424S-MN} | 10⁷ | 8 | 0 | 8 | 100 | 28.0 (0.0) | — | — | — |
| 6 | F1-V_{STD} | 10⁸ | 7 | 3 | 10 | 70 | 21.9 (3.6) | 7.7 (2.1) | 8 | 10 |
| 7 | F1-V_{MN} | 10⁸ | 8 | 2 | 10 | 80 | 23.1 (4.4) | 3.5 (2.1) | 2 | 5 |
| 8 | F1-V_{AG} | 10⁸ | 8 | 2 | 10 | 80 | 23.4 (4.1) | 5.0 (2.8) | 3 | 7 |
| 9 | F1-V_{C424S-MN} | 10⁸ | 9 | 0 | 9 | 100 | 28.0 (0.0) | — | — | — |
| 10 | F1-V_{STD} | 10⁹ | 5 | 5 | 10 | 50 | 17.9 (3.6) | 7.8 (3.3) | 4 | 13 |
| 11 | F1-V_{MN} | 10⁹ | 4 | 4 | 8 | 50 | 17.1 (4.6) | 6.3 (4.9) | 2 | 11 |
| 12 | F1-V_{AG} | 10⁹ | 3 | 7 | 10 | 30 | 13.4 (3.8) | 7.1 (3.2) | 3 | 11 |
| 13 | F1-V_{C424S-MN} | 10⁹ | 4 | 6 | 10 | 40 | 16.2 (3.4) | 8.3 (3.2) | 3 | 12 |

Pairwise Comparison p-values by Challenge Dose

| | Percent Survival | | | Time to Death | | |
|---|---|---|---|---|---|---|
| Comparison Groups | 10⁷ | 10⁸ | 10⁹ | 10⁷ | 10⁸ | 10⁹ |
| F1-V_{STD} vs. F1-V_{MN} | 1.0000 | 1.0000 | 1.0000 | 0.5881 | 0.0220 | 0.6349 |
| F1-V_{STD} vs. F1-V_{AG} | 1.0000 | 1.0000 | 0.8062 | 0.0153 | 0.1360 | 0.6800 |
| F1-V_{STD} vs. F1-V_{C424S-MN} | 0.5437 | 0.5118 | 0.9655 | ** | ** | 0.9600 |
| F1-V_{STD} vs. ALH | 0.0009 | 0.0015 | 0.0076 | 0.0010 | 0.0019 | 0.0126 |
| F1-V_{MN} vs. F1-V_{AG} | 1.0000 | 1.0000 | 0.8948 | 0.0423 | 0.6031 | 0.9798 |
| F1-V_{MN} vs. F1-V_{C424S-MN} | 0.8351 | 0.8044 | 1.0000 | ** | ** | 0.7977 |
| F1-V_{MN} vs. ALH | 0.0003 | 0.0004 | 0.0248 | 0.0032 | 0.3795 | 0.0426 |
| F1-V_{AG} vs. F1-V_{C424S-MN} | 0.8354 | 0.8044 | 1.0000 | ** | ** | 0.9349 |
| F1-V_{AG} vs. ALH | 0.0001 | 0.0004 | 0.1776 | 0.1534 | 0.1296 | 0.0126 |
| F1-V_{C424S-MN} vs. ALH | <.0001 | <.0001 | 0.0717 | ** | ** | 0.0035 |

### Example 16

### Summary

This Example presents a summary of the results disclosed above. The 53-kDa F1-V fusion protein was modified by site-directed mutagenesis to replace the sole cysteine with a serine residue, thus producing F1-V_{C424S}. Novel F1-V purification methods were employed to isolate monomeric F1-V and F1-V_{C424S} that resulted in 1 to 2 mg of >95% pure, mono-disperse protein per gram of cell paste. Standard (cysteine containing) F1-V and F1-V_{C424S} were compared for stability and aggregation characteristics under various conditions of solution pH and buffer additive. Predominately monomeric F1-V forms were observed at pH 10.0 with progressive aggregation occurring as pH conditions were lowered toward pH 5.0. Of the buffer additives that were compared, L-cysteine was found to provide the best disulfide bond disruption, while L-arginine (Tsumoto et al., (2004) Biotechnol. Prog. 20, pp. 1301-1308) was found to be the most effective additive for disrupting non-covalent multimer associations.

Standard, cysteine-containing F1-V formulations were evaluated side-by-side with the modified F1-V_{C424S} form for protective efficacy against lethal plague challenge in mice. Thus, substitution of the cysteine residue with serine did not statistically affect the activity of F1-V to elicit protective immunity against plague. Moreover, the monomeric and multimeric forms of F1-V exhibit equivalent immunogenicity and protective efficacy against subcutaneous infection.

Numerous expression and purification strategies for F1-V have been published ranging from traditional prokaryotic systems (Heath et al., (1998) Vaccine 16, pp. 1131-1137; Powell et al., (2005) Biotechnol. Prog. 21 (2005), pp. 1490-1510; Williamson, (2001) J. Appl. Microbiol. 91, pp. 606-608; Andrews et al., (1996) Infect. Immun. 64, pp. 2180-2187) to transgenic tomatoes (Alvarez et al., (2006) Vaccine 24, pp. 2477-2490) and the tobacco-like Nicotiana benthamiana (Santi et al., (2006) Proc. Natl. Acad. Sci. USA 103, pp. 861-866). Regardless of the ultimate expression strategy employed, the final F1-V fusion protein will retain a tendency to multimerize because of its subunit composition. Although this self-association is due mainly to the F1 subcomponent, the fusion architecture actually reduces polydispersity compared to the individual F1 protein, which is even more aggregative (Powell et al., (2005) Biotechnol. Prog. 21 (2005), pp. 1490-1510). Thus, the F1-V fusion based plague antigen is at the forefront of plague vaccine development (Glynn et al., (2005) Vaccine 23, pp. 1957-1965; Tripathi et al., (2006) Vaccine 24, pp. 3279-3289; Titball et al., (2004) Expert Opin. Biol. Ther. 4, pp. 965-973; Leary et al., (1997) Microb. Pathog. 23, pp. 167-179). As demonstrated herein, use of the described F1-V_{C424S} protein form facilitates the enhanced production and stability of F1-V-based plague vaccines.

### Example 17

### Administration of F1-V_{C424S} to a Human Subject

This Example demonstrates a method of administering F1-V_{C424S} to a subject. A suitable subject for receiving the F1-V_{C424S} vaccine is one who is at risk for exposure to Y. pestis bacteria, for instance a member of the military who may be at risk for exposure to bioweapons. In some embodiments, a Y. pestis titer is taken prior to vaccine administration to determine whether the subject has been exposed previously to the bacteria.

The F1-V_{C424S} vaccine is provided as an aluminum hydroxide adjuvant-adsorbed pharmaceutical composition, and is administered subcutaneously in a dose that includes about 0.1 μg to 10 mg of immunogenic F1-V_{C424S} protein. A second dose is administered in the same fashion approximately three months after the first dose, and the efficacy of protection against Y. pestis infection is assessed by measuring antibody titers using standard laboratory protocols.

While this disclosure has been described with an emphasis upon particular embodiments, it will be obvious to those of ordinary skill in the art that variations of the particular embodiments can be used and it is intended that the disclosure can be practiced otherwise than as specifically described herein. Accordingly, this disclosure includes all modifications encompassed within the spirit and scope of the disclosure as defined by the following claims:

We claim:

1. An isolated immunogenic protein comprising a substantially monodisperse F1-V fusion protein.

2. The isolated immunogenic protein of claim 1, wherein the protein comprises:
(a) about 50% monodisperse F1-V fusion protein;
(b) about 60% monodisperse F1-V fusion protein;
(c) about 70% monodisperse F1-V fusion protein;
(d) about 80% monodisperse F1-V fusion protein;
(e) about 90% monodisperse F1-V fusion protein; or
(f) about 100% monodisperse F1-V fusion protein.

3. The isolated immunogenic protein of claim 1, wherein the F1-V fusion protein comprises:
(a) an amino acid sequence set forth as SEQ ID NO: 1, wherein Xaa at position 424 is cysteine, methionine, serine, glycine, glutamic acid, aspartic acid, valine, threonine, tyrosine, or alanine; or
(b) an amino acid sequence having at least 95% sequence identity with (a).

4. The isolated immunogenic protein of claim 3, wherein Xaa at position 424 is methionine, serine, glycine, glutamic acid, aspartic acid, valine, threonine, tyrosine, or alanine.

5. The isolated immunogenic protein of claim 4, wherein Xaa at position 424 is serine.

6. The isolated immunogenic protein of claim 3, wherein Xaa at position 150 is glutamic acid or asparagine.

7. The isolated immunogenic protein of claim 3, wherein Xaa at position 151 is phenylalanine, methionine, leucine, or tyrosine.

8. The isolated immunogenic protein of claim 3, wherein Xaa at position 150 is glutamic acid, and wherein Xaa at position 151 is phenylalanine.

9. The isolated immunogenic protein of claim 1 comprising an amino acid sequence set forth as SEQ ID NO: 2.

10. The isolated immunogenic protein of claim 1 consisting of an amino acid sequence set forth as SEQ ID NO: 2.

11. An isolated polynucleotide comprising a nucleic acid sequence encoding the immunogenic protein of claim 3.

12. The polynucleotide of claim 11, operably linked to a promoter.

13. A vector comprising the polynucleotide of claim 11.

14. The isolated immunogenic protein of claim 1, wherein the protein provides protective immunity from Y. pestis when administered to a subject in a therapeutically effective amount.

15. A pharmaceutical composition comprising the immunogenic protein of claim 1 and a pharmaceutically acceptable carrier.

16. The composition of claim 15, wherein the composition is adsorbed to an aluminum hydroxide adjuvant.

17. The composition of claim 15, wherein the composition comprises from about 0.5 mM L-cysteine to about 5 mM L-cysteine.

18. The composition of claim 15, wherein the composition comprises from about 0.06 M L-arginine to about 6 M L-arginine.

19. The composition of claim 15, further comprising a therapeutically effective amount of IL-2, GM-CSF, TNF-α, IL-12, and IL-6.

20. A method for eliciting an immune response in a subject, comprising:
(a) selecting a subject in which an immune response to the immunogenic protein of claim 1 is desirable; and
(b) administering to the subject a therapeutically effective amount of the immunogenic protein of claim 1, thereby producing an immune response in the subject.

21. The method of claim 20, wherein administration comprises oral, topical, mucosal, or parenteral administration.

22. The method of claim 21, wherein parenteral administration comprises intravenous administration, intramuscular administration, or subcutaneous administration.

23. The method of claim 20, wherein administration comprises from about one to about six doses.

24. The method of claim 23, wherein administration comprises two doses.

25. The method of claim 20, further comprising administering an adjuvant to the subject.

26. The method of claim 20, further comprising administering to the subject a therapeutically effective amount of IL-2, RANTES, GM-CSF, TNF-α, IFN-γ, G-CSF or a combination thereof.

27. A method of inhibiting Yersinia pestis infection in a subject, the method comprising:
(a) selecting a subject at risk for exposure to Yersinia pestis; and
(b) administering to the subject a therapeutically effective amount of the immunogenic protein of claim 1, thereby inhibiting Yersinia pestis infection in the subject.

28. A method of making the isolated substantially monodisperse immunogenic protein of claim 1, wherein the method comprises ion exchange chromatography, and wherein the ion exchange chromatography dilution buffer comprises guanidine HCl.

29. The method of claim 28, wherein the ion exchange chromatography dilution buffer comprises from about 3 M guanidine HCl to about 9 M guanidine HCl.

30. The method of claim 28, wherein the immunogenic protein is precipitated at a pH of about 4.7-5.2.

31. The method of claim 30, wherein the method further comprises raising the pH of the immunogenic protein to about 7.8-11.0.

32. The method of claim 29, wherein the method further comprises hydroxyapatite chromatography.

33. The method of claim 29, wherein the hydroxyapatite comprises ceramic hydroxyapatite or fluoroapatite.


SEQUENCE LISTING (7 sequences)

SEQ ID NO: 1 - protein, 478 aa, Y. pestis
  misc_feature (151)..(152): Xaa can be any naturally occurring amino acid
MADLTASTTATATLVEPARITLTYKEGAPITIMDNGNIDTELLVGTLTLGGYKTGTTSTSVNFTDAAGDPMYLTFTSQDGNNHQFTTKVIGKDSRDFDISPKVNGENLVGDDVVLATGSQDFFVRSIGSKGGKLAAGKYTDAVTVTVSNQXXMIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTXSDKSRPLNDLVSQKTTQLSDITSRFNSAIEALNRFIQKYDSVMQRLLDDTSGK

SEQ ID NO: 2 - protein, 478 aa, Y. pestis
MADLTASTTATATLVEPARITLTYKEGAPITIMDNGNIDTELLVGTLTLGGYKTGTTSTSVNFTDAAGDPMYLTFTSQDGNNHQFTTKVIGKDSRDFDISPKVNGENLVGDDVVLATGSQDFFVRSIGSKGGKLAAGKYTDAVTVTVSNQEFMIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTSSDKSRPLNDLVSQKTTQLSDITSRFNSAIEALNRFIQKYDSVMQRLLDDTSGK

SEQ ID NO: 3 - DNA, 39 bp, Artificial sequence
  primer
ctcactttgccaccacctcctcggataagtccaggccgc

SEQ ID NO: 4 - DNA, 39 bp, Artificial sequence
  Primer
gcggcctggacttatccgaggaggtggtggcaaagtgag

SEQ ID NO: 5 - protein, 170 aa, Y. pestis
MKKISSVIAIALFGTIATANAADLTASTTATATLVEPARITLTYKEGAPITIMDNGNIDTELLVGTLTLGGYKTGTTSTSVNFTDAAGDPMYLTFTSQDGNNHQFTTKVIGKDSRDFDISPKVNGENLVGDDVVLATGSQDFFVRSIGSKGGKLAAGKYTDAVTVTVSNQ

SEQ ID NO: 6 - protein, 2 aa, Artificial sequence
  Spacer
EF

SEQ ID NO: 7 - protein, 324 aa, Y. pestis
MIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGK